(19) United States
(12) Patent Application Publication
(10) Pub. No.: US 2013/0040310 A1
(43) Pub. Date: Feb. 14, 2013

(54) ULK1 COMPOSITIONS, INHIBITORS, SCREENING AND METHODS OF USE

(71) Applicants: Reuben Shaw, San Diego, CA (US); Daniel F. Egan, San Diego, CA (US); Maria Mihavlova, San Diego, CA (US); David Shackelford, Redondo Beach, CA (US)

(72) Inventors: Reuben Shaw, San Diego, CA (US); Daniel F. Egan, San Diego, CA (US); Maria Mihavlova, San Diego, CA (US); David Shackelford, Redondo Beach, CA (US)

(73) Assignee: Salk Institute for Biological Studies, La Jolla, CA (US)

(21) Appl. No.: 13/516,343

(22) PCT Filed: Dec. 15, 2010
(86) PCT No.: PCT/US10/60578

§ 371 Date: Sep. 10, 2012

Related U.S. Application Data
(60) Provisional application No. 61/286,733, filed on Dec. 15, 2009.
(60) Provisional application No. 61/325,361, filed on Apr. 18, 2010.

(51) Int. Cl. C07K 7/06; C12N 5/09; C07K 16/40; C07K 7/08; C07K 14/47; C07K 19/00; G01N 33/574; C12Q 1/48

(52) U.S. Cl. 435/7.4; 435/375; 435/15; 530/328; 530/327; 530/326; 530/325; 530/324; 530/300; 530/350; 530/387.9

(57) ABSTRACT

This disclosure relates to methods and compositions useful for the treatment of cancer and diseases and disorders associated with autophagy.

### CROSS REFERENCE TO RELATED APPLICATIONS

This application claims priority under 35 U.S.C. §119 from Provisional Application Ser. No. 61/325,361, filed Apr. 18, 2010, the disclosure of which is incorporated herein by reference. The disclosure of provisional application No. 61/286,733, filed Dec. 15, 2009, is incorporated herein for all purposes.

### TECHNICAL FIELD

The disclosure relates to methods for screening, methods of treatment and diagnosis and methods for targeted pharmaceutical development of ULK1 agents.

### BACKGROUND

Autophagy is a degradative process that recycles long-lived and faulty cellular components. It is linked to many diseases and is required for normal development.

### SUMMARY

AMPK is a conserved sensor of intracellular energy activated in response to low nutrients and a variety of environmental stresses. In response to lowered ATP levels, AMPK serves as a metabolic checkpoint, reducing biosynthetic catabolic processes while upregulating anabolic processes to restore energy levels in the cell. A cellular mechanism for adapting to loss of nutrients and biosynthetic intermediates is the process of autophagy, by which cells cannibalize their organelles and proteins through a highly conserved genetic pathway whose biochemical details remain mostly unknown in mammalian cells. Despite an obvious need for coordination of autophagy under conditions of low energy, no mechanistic connection between AMPK and the autophagic machinery has been discovered to date. The disclosure demonstrates that the mammalian orthologs of the yeast kinase Atg1, ULK1 and UKL2 are direct substrates of AMPK and that these substrates regulate autophagy induction under conditions of low energy. Genetic analysis of AMPK or ULK1 in mammalian liver as well in C. elegans reveals a conserved role for these kinases in autophagy initiation, and profound defects in their absence. Most notably, loss of AMPK or ULK1 results in dramatic accumulation of the autophagy adaptor p62/SQSTM1 and defective mitophagy. Reconstitution of ULK1/2 deficient cells with a mutant ULK1 that cannot be phosphorylated by AMPK reveals that AMPK phosphorylation of ULK1 is required for induction of autophagy. These findings uncover a conserved biochemical mechanism coupling nutrient status with growth control and autophagy initiation.

The disclosure provides an isolated polypeptide comprising a fragment of SEQ ID NO:2 containing a phosphorylatable domain containing the sequence of SEQ ID NO:3, 4, 5, 6, 7, 8, 9 or 10, wherein the polypeptide lacks ULK1 activity. In one embodiment, the polypeptide contains the sequence of SEQ ID NO:3, 4, 5, or 6. In yet another embodiment, the polypeptide consisting of about 10-100 amino acids and having a phosphorylatable domain containing the sequence of SEQ ID NO:3, 4, 5, or 6. In yet another embodiment, the polypeptide is about 10-50 amino acids in length. In yet another embodiment, the polypeptide is about 10-30 amino acids in length. In yet another embodiment, the polypeptide is about 10-20 amino acids in length. In yet another embodiment, the polypeptide is about 10 amino acids in length. In yet another embodiment, the polypeptide contains at least one unnatural amino acid or D-amino acid.

The disclosure also provides a fusion polypeptide comprising a first domain comprising a polypeptide as described above and a second domain comprising a domain of interest. In one embodiment, the second domain is a protein transduction domain. In another embodiment, the second domain is a receptor ligand domain.

The disclosure also provides a method of screening for an agent that modulates autophagy or energy metabolism comprising contacting a phosphorylatable polypeptide of the disclosure comprising a consensus sequence of SEQ ID NO:7, 8, 9 or 10 with an agent in the presence of an AMPK polypeptide and determining whether the polypeptide is phosphorylated or dephosphorylated, wherein a change in phosphorylation compared to the polypeptide in the presence of AMPK but in the absence of the agent is indicative of an agent that modulates autophagy. In one embodiment, the agent is selected from the group consisting of a peptide, peptidomimetic, polypeptide, antibody, antibody fragment or small molecule.

The disclosure also provides an isolated phosphorylation site-specific antibody that specifically binds to a human ULK1 polypeptide antigenic domain at a site containing a sequence as set forth in SEQ ID NO:7, 8, 9 or 10. In one embodiment, the phosphorylation site-specific antibody specifically binds a human ULK1 polypeptide at a sequence selected from the group consisting of SEQ ID NO:3, 4, 5 or 6, wherein said antibody binds said polypeptide when phosphorylated at the serine of SEQ ID NO:3, 4 or 6 or the threonine of SEQ ID NO:5. In one embodiment, the antibody binds to the sequence of SEQ ID NO:4 and the phosphorylated residue is the serine.

The disclosure also provides an isolated phosphorylation site-specific antibody that specifically binds a human ULK1 polypeptide at a sequence selected from the group consisting of SEQ ID NO:3, 4, 5, or 6, wherein said antibody binds said polypeptide when not phosphorylated at the serine of SEQ ID NO:3, 4, or 6 or the threonine of SEQ ID NO:5.

The disclosure also provides a method of detecting a cancer associated with aberrant autophagy comprising contacting a cancer tissue sample with an antibody the specifically binds to a sequence set forth in SEQ ID NO:3, 4, 5, 6, 7, 8, 9 or 10 and determining whether the antibody binds to a ULK polypeptide in the sample, wherein binding of a non-phosphorylated ULK1 is indicative of a cancer associated with aberrant autophagy.

The disclosure also provides a method of increasing a cellular response to a cancer therapy comprising inhibiting phosphorylation or activity of ULK1 in a cell currently undergoing the cancer therapy. In one embodiment, the inhibiting comprises inhibiting phosphorylation at a site containing a sequence selected from the group consisting of SEQ ID NO: 3, 4, 5, 6, 7, 8, 9 or 10. In one embodiment, the method comprises contacting a cancer cell with a polypeptide of the disclosure containing a sequence of SEQ ID NO:3, 4, 5, 6, 7, 8, 9 or a combination thereof. In another embodiment, the cancer therapy comprises endocrine therapy, chemotherapy or radiation therapy.

The disclosure also provides a method of treating, inhibiting or preventing a cancer, type II diabetes, inflammatory disease or mental disease or disorder associated with protein misfolding and aggregation comprising contacting a cell with an agent that promotes phosphorylation of ULK1 at a site containing a sequence selected from the group consisting of 3, 4, 5, 6, 7, 8, 9 or 10. In one embodiment, the cancer is breast cancer, liver cancer, ovarian cancer, gastric cancer, bladder cancer, colon cancer, prostate cancer, lung cancer, nasopharyngeal carcinoma, cervical carcinoma, skin cancer, brain cancer, neuroblastoma, glioma, a solid tumour, a hematologic malignancy, leukemia, lymphoma, or head and neck cancer. In another embodiment, the mental disease or disorder associated with protein misfolding or aggregation is selected from the group consisting of Alzheimer disease, Parkinson disease, tauopathies, and polyQ3 expansion diseases.

The disclosure also provides a pharmaceutical composition comprising a modulator of phosphorylation of ULK 1 at a site containing the sequence of SEQ ID NO: 3, 4, 5, 6, 7, 8, 9 or 10.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A-F shows ULK1 is a conserved substrate of AMPK. (A) Clustal alignment of four conserved sites in ULK1 and two sites in ULK2 matching the optimal AMPK substrate motif. (B) ULK1 and GST or GST-14-3-3 expression vectors were transfected into human embryonic kidney (HEK) 293T cells, and placed in media containing 20 μM STO-609 (STO), vehicle (veh), or 5 mM phenformin (Phen) for 1 h. Cell lysates and GST pulldowns were immunoblotted as indicated. (C) In vitro kinase assays with myc-tagged catalytically inactive (KI: K46I) ULK1 or myctagged wild-type raptor which were immunoprecipitated from HEK293T cells and used as substrates for purified active AMPK in the presence of ³²P-γ-ATP. (D) HEK293T cells transfected with myc-tagged wild-type ULK1 or indicated Serine-to-alanine ULK1 mutants were treated with either vehicle or 1 mM phenformin for 1 h, or were co-transfected with a constitutively active AMPKα1 (aa1-312) mammalian expression vector (11). Proteins from lysates were immunoblotted with phospho-specific antibodies as indicated. (E) In vitro kinase assays using myc-ULK1 and purified AMPK as above. Phosphorylation of myc-ULK1 detected by immunoblotting with indicated phospho-specific antibodies. (F) Primary murine embryonic fibroblasts (MEFs) were treated with 2 mM AICAR or vehicle for 1 h. Lysates immunoblotted as indicated including detection of endogenous ULK1 P-Ser555.

FIG. 2A-D shows genetic deficiency for AMPK or ULK1 in murine liver or primary murine hepatocytes results in autohagy defects. (A) Liver lysates from littermate-matched mice were immunoblotted for the indicated antibodies. p62 to actin ratio calculated from densitometry performed on immunoblots. Data shown as mean+/−SEM. * p<0.01 (B) Primary hepatocytes derived from ULK1^{+/+} or ULK1^{-/-} mice or AMPKα1^{+/-} a2^{L/+} or AMPKα1^{-/-} a2^{L/L} as described in methods were placed in media containing 2 mM metformin (met) or vehicle (veh) for 2 h. Lysates were immunoblotted with the indicated antibodies. (C) Transmitting electron microscopy (TEM) was performed on primary murine hepatocytes of the indicated genotypes revealing accumulation of mitochondria in both AMPK- and ULK1-deficient cells. Mitochondria pseudocolored RED, cytoplasm BLUE, nuclei GREEN, and lipid droplets YELLOW. (D) Primary murine hepatocytes of the indicated genotypes stained by immunocytochemistry for the mitochondrial marker TOM20 (red) and nuclei (blue). Scale bar, 10 microns.

FIG. 3A-D shows AMPK is necessary and sufficient for autophagy induction in C. elegans. (A) Insulin receptor daf-2(e1370) mutant worms expressing GFP::LGG-1 (equivalent to GFP-LC3) were treated with control RNAi or RNAi against bec-1 (Beclin), aak-2 (AMPKα2) or unc-51 (ULK1) and the number of LGG-1/LC3-positive puncta per hypodermal seam cell were quantified. (B) aak-2 (ok524) mutants or wild-type N2 (WT) animals expressing GFP::LGG-1 were treated with control or daf-2 RNAi and were scored for LGG-1 positive puncta per seam cell. (C) Transgenic worms expressing constitutively active AAK-2 (amino acids 1-321)::TOMATO (CAAAK-2::TOMATO (1-321) fusion or controls were analyzed for LGG-1 positive puncta per seam cell. (D) Animals expressing both CA-AAK-2 (1-321)::TOMATO and GFP::LGG-1 were treated with control or unc-51 RNAi and scored for LGG-1/LC3-positive puncta per seam cell. All panels show relative counts, see FIG. 14 for details. Data shown as mean+/−SEM. * P<0.0001.

FIG. 4A-G shows AMPK phosphorylation of ULK1 is required for mitophagy and cell survival upon nutrient deprivation (A) U2OS cells stably expressing mouse wild-type (WT) or catalytically inactive (KI) or AMPK non-phosphorylatable (4SA) ULK1 cDNA or the empty retroviral vector (v) along with a shRNA against endogenous human ULK1 and ULK2 were placed in media containing 5 mM phenformin (Phen) or vehicle for 1 h. Lysates were immunoblotted as indicated. (B) ULK1−/− MEFs stably expressing WT, KI, or 4SA ULK1 cDNA or the empty retroviral vector (v) along with a shRNA against endogenous ULK2 were placed in EBSS starvation media (starv) or control media (ctl) for 6 h in the presence or absence of BafilomycinA (BafA) and immunoblotted as indicated. (C) Cells from (B) analyzed by TEM and Inform morphometric software. Mitochondria pseudocolored RED, cytoplasm BLUE, and nuclei GREEN. (D) Flourescence Activated Cell Sorting (FACS) analysis on cells from (B) which were stained with JC-1 under basal conditions, or with the mitochondrial uncoupler CCCP as a control, to measure mitochondrial membrane potential. Compromised mitochondrial membrane potential is shifted to the left, as observed in cells treated with CCCP. (E) Wild-type (WT) MEFs transfected with 20 nM siRNA pools to a universal control (ctl), murine Atg5, or murine ULK1 and ULK2 for 72 hours were then placed in starvation medium (starv) or standard media (ctl) for 12 h and cell death was scored by AnnexinV-FACS. (F) Cells from (B) were placed in starvation medium (starv) or standard media (ctl) for 12 h and cell death was scored by AnnexinV-FACS. (G) Model for AMPK activation of ULK1 in a two-pronged mechanism via direct phosphorylation of ULK1 and inhibition of mTORC1 suppression of ULK1.

FIG. 5 shows ULK1 and ULK2, but not ULK3 co-immunoprecipitate with endogenous AMPK. Immunoprecipitates of FLAG-tagged ULK1, ULK2, ULK3 or empty FLAG-tagged vector (vec) transfected in HEK-293T cells were immunoblotted for endogenous AMPK subunits as indicated. Data is representative of 3 independent experiments.

FIG. 6 shows identification of endogenous AMPK as an interacting partner of ULK2 Eluted FLAG-tagged ULK2 immunoprecipitates from HEK-293T cells were resolved on SDSPAGE and lanes 1 (mock transfected) and 3 (ULK2) of SuproRuby stained gel shown were sliced into 25 fragments each and analyzed by tandem mass spectrometry. Endogenous FIP200 as well as the indicated AMPK subunits were uniquely found in the ULK2 immunoprecipitates and bands corresponding to each in the ULK2 lanes are indicated. Mascot scores are indicated as well as number of unique peptides analyzed.

FIG. 7 is a diagram of all LC/MS/MS identified in vivo phosphorylation sites in human ULK1. Top: Myc-ULK1 was transfected into human embryonic kidney (HEK)-293T cells, treated with 5 mM phenformin to reduce cellular ATP for 1 hour and immunoprecipitated with anti myc antibody. The IP was run out on SDS PAGE, stained with coomassie, and the band corresponding to myc-ULK1 was cut out, and isolated and subjected to tryptic digest and LC/MS/MS analysis. Three indicated sites matching the AMPK substrate motif were identified, all mapping to the serine—rich unstructured region between the N-terminal kinase domain and the conserved C-terminus that mediates ULK1 binding to its subunits Atg13 and FIP200. Bottom: Quantitative mass spec spectrometry (MS/MS TIC ratio quantification) reveals that tryptic peptides containing Phospho-Ser555 and Phospho-Ser637 of ULK1 are induced by treatment of HEK-293T cells with 5 mM phenformin for 1 h.

FIG. 8 shows a schematic of potential AMPK-dependent phosphorylation sites in ULK1.

FIG. 9 shows myc-tagged ULK1 is phosphorylated by exogenous AMPK in vitro. In vitro kinase assays (from FIG. 1C) using purified Myc-tagged kinase-inactive (KI: K46I) ULK1 or myc-tagged wild-type raptor as substrates. Myc-tagged proteins (1-2 ug per r×n) were produced in HEK-293T cells, purified, and subjected to an in vitro kinase assay in the presence of radioactive ³²P-γ-ATP with or without recombinant active heterotrimeric AMPK added as indicated (0.1 U per r×n). The kinase assays were resolved and parallel non-radioactive kinase assays were immunoblotted with AMPK alpha or myc-tag antibodies as indicated. 32P signal was captured on a phosphoimager and its signal, as well as that of the anti-myc immunoblot, was densitometrically quantified and expressed as a ratio of ³²P incorporated per mol myctagged protein.

FIG. 10 shows endogenous phospho ULK1 Ser555 increases with AICAR in primary MEFs. Endogenous ULK1 is phosphorylated on Ser555 in an AMPK-dependent manner following 2 mM AICAR treatment for 1 h in primary murine embryonic fibroblasts (MEFs). Phospho-ULK1, Phospho-ACC and total ULK1 and total ACC were quantified by densitometry and their ratios graphed. Data in all experiments is representative of 3 independent experiments.

FIG. 11 shows p62 and ubiquitin are elevated in AMPK deficient livers compared to littermate controls as visualized by immunohistochemistry. AMPK deficient (a1−/−; a2lox/lox; tail-vein adenovirus cre-injected) livers or control (a1+/−; a2lox/+; tail vein adenovirus cre injected) livers were subjected to immunohistochemistry for the p62 autophagy marker or ubiquitin. Note elevated p62 and ubiquitin levels in the AMPK double knockout (DKO) liver. Overlapping aggregates of p62 and ubiquitin from serial sections indicated with red arrowheads. H&E is shown illustrating normal liver architecture. Data are representative of 2 independent experiments and 5 mice of each genotype analyzed.

FIG. 12 shows p62 and the mitochondrial protein COXIV are elevated with loss of ULK1 or AMPK in primary murine hepatocytes. Primary hepatocytes were derived from ULK1^{+/+} or ULK1^{-/-} mice or aforementioned AMPK mice 7 days after tail-vein injection of adenovirus-cre. Cells were treated with 2 mM metformin (met) or vehicle (veh) for 2 h and immunoblotted with the indicated antibodies including p62 and the mitochondrial marker CoxIV, which are densitometrically quantified relative to actin below. Data is representative of 3 independent experiments.

FIG. 13 shows the relative number of mitochondria are increased with loss of ULK1 or AMPK in primary murine hepatocytes. Quantification of TEM from primary murine hepatocytes from FIG. 2C. Top: boxwhisker plot of number of mitochondria per cell from 5 independent TEM fields. Below: The number of mitochondria per cell as expressed relative to levels seen in littermate matched control hepatocytes (set to 1.0). Data shown as mean+/−SEM. *P<0.01 student's unpaired t-test.

FIG. 14 shows analysis of strains expressing the transgene LGG-1::GFP. Summary of LGG-1::GFP positive puncta in L3 larvae of different genetic backgrounds using the LGG-1::GFP reporter. The average number of puncta per seam cell was calculated from two-three independent trials. N-seam cells, number of seam cells analyzed, N-animals, number of animals in which seam cells were analyzed. P values were calculated as unpaired, two-tailed t-test. N/A, animals were grown on regular OP50 E. coli bacteria. “hets” refer to F1 animals analyzed from cross between DA2123 and AGD383. Animals were raised at 20°.

FIG. 15 shows validation of lentiviral shRNAs against human and murine ULK1 and ULK2. Top: Quantitative RT-PCR was used to validate the effectiveness of lentiviruses bearing hairpin shRNAs against murine and human ULK2 in cell types indicated. mRNA levels were normalized to GAPDH mRNA levels. Bottom: Immunoblotting was used to validate the effectiveness of lentiviruses bearing hairpin shRNAs against human ULK1. Lentivirus shRNAs A8/B10 directed against AMPKalpha served as a positive control for viral tittering and LKO is the empty lentiviral vector negative control. Raptor served as a loading control here.

FIG. 16 shows U2OS cells lacking ULK1/2 function is mirrored by mutation of the AMPK sites in ULK1 and these sites are required for ULK1 function. U2OS cells were stably infected with empty lentiviral vector pLKO (vec) or human ULK1 and ULK2 shRNA-expressing lentiviruses and then stably reconstituted with retroviruses bearing wild-type (WT) or kinase-inactive (KI) or AMPK non-phosphosphorylatable (4SA) ULK1 cDNA or the empty retroviral vector (v). WT ULK1, but not KI or 4SA ULK1 was able to restore p62 degradation to the ULK1/2-deficient U2OS cells, which is quantified by densitometry. Cells were treated with 5 mM Phenformin (Phen) or vehicle for 1 h. Results are representative of 3 independent experiments.

FIG. 17 shos the reconstitution strategy for ULK1^{-/-} MEFs. Retroviruses bearing myc-tagged wild-type or kinase inactive or non-phosphorylatable ULK1 were introduced into ULK1^{-/-} MEFs and after selection these cells were next infected with lentiviruses bearing shRNAs for murine ULK2. The same strategy was used for replacing ULK1/2 function in U2OS cells except there after the retroviral selection, cells were co-infected with lentiviruses bearing shRNAs against human ULK1 and ULK2.

FIG. 18 shows MEFs lacking ULK1/2 function is mirrored by mutation of the AMPK sites in ULK1 and these sites are required for ULK1 function. ULK1^{-/-} MEFs bearing stable murine ULK2 shRNA lentiviruses were stably reconstituted with retroviral vectors bearing WT, KI, or 4SA ULK1 or no cDNA (vec) and then placed in starvation media (stary—EBSS: Earle's buffered salt solution) or control media (ctl:standard media for these cells: DMEM+10% FBS) for 6 h in the presence of BafilomycinA (BafA) as indicated. Lipidated LC3 (II) and p62 levels were quantified by densitometry and shown at bottom. Results are representative of 3 independent experiments.

FIG. 19 shows measurements of LC3 punctae using ImageJ macro morphometric software. Starvation in EBSS media in our MEF cell model induces endogenous LC3 punctae which can be measured and quantified using anti LC3 antibody (Cell Signaling #3868) and subsequent ImageJ quantification of immunocytochemistry. Data representative of cells from >ten 63× fields.

FIG. 20 shows MEFs lacking ULK1/2 function is mirrored by mutation of the AMPK sites in ULK1 with regard to endogenous LC3 staining after starvation. Localization of endogenous LC3 in 6 h starved (EBSS) BafA treated cells from FIG. 4B. LC3 was localized to puncta following placement of MEF cells into starvation medium. LC3 positive puncta were quantified using an LC3 ImageJ macro as outlined in FIG. 19. Ten 63× fields per condition were quantified. In contrast to p62, ULK1-defiency did not prevent endogenous LC3 lipidation or puncta formation in this cell type, consistent with findings that neither ULK1-nor AMPK-deficiency impact LC3 conversion in MEFs.

FIG. 21 shows Mitotracker staining of ULK1 cDNA-reconstituted ULK1^{-/-} Mitotracker Red CMXRos staining of mitochondria used at 50 nM for 15 min. Images from WT, KI, or 4SA reconstituted ULK1^{-/-} MEFs bearing ULK2 shRNA taken on confocal microscope.

FIG. 22 shows the relative number of mitochondria are increased with loss of ULK1/2 function. Quantification of the number of mitochondria per cell from FIG. 4C as analyzed using Inform morphometric software as in FIG. 12. Cells expressing WT ULK1 were set to 1.0 (=avg 32.5 mito/cell).

FIG. 23 shows mitochondrial defects in ULK-deficient MEFs reconstituted with KI or 4SA mutant ULK1. Transmitting Electron Microscopy images from WT, KI, or 4SA reconstituted ULK1^{-/-} MEFs bearing ULK2 shRNA. Cells were placed into fresh growth media (ctl) or EBSS (starvation) at time zero and fixed at 4 h. Note the aberrant elongated mitochondria in the KI and 4SA cells, and the altered mitochondrial cristae in the KI or 4SA reconstituted cells following starvation. Images taken at 13,500×.

FIG. 24 shows validation of Dharmacon smartpool siRNAs against murine ULK1, ULK2, and Atg5. Wild-type (WT) MEFs transfected with 20 nM siRNA pools to a universal control (ctl), murine Atg5, or murine ULK1 and ULK2 for 72 hours. Proteins from lysates were immunoblotted with indicated total antibody.

FIG. 25 shows a model for AMPK and mTOR regulation of the activity of the ULK1 complex via opposing phosphorylation events. Schematic of intersection of mTOR, AMPK, and ULK1 pathways. Top: Under nutrient replete conditions. Bottom: Under nutrient deprived conditions.

### DETAILED DESCRIPTION

As used herein and in the appended claims, the singular forms “a,” “and,” and “the” include plural referents unless the context clearly dictates otherwise. Thus, for example, reference to “a protein” includes a plurality of such proteins and reference to “the cell” includes reference to one or more cells known to those skilled in the art, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

The publications discussed above and throughout the text are provided solely for their disclosure prior to the filing date of the present application. Nothing herein is to be construed as an admission that the inventors are not entitled to antedate such disclosure by virtue of prior disclosure.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of”.

One aspect of successful treatment of various diseases and disorders includes identification of the cellular basis of the disease or disorder. For example, successful treatment of cell proliferative disorders including, but not limited to, cancers and neoplasms, requires the identification of drug therapies for the specific biological cause of the cell proliferative disorders.

For example, identification of cell proliferative disorders caused by kinases will assist in identifying those cell proliferative diseases and disorders that are likely to be responsive to a particular inhibitor drug. Various kinase inhibitors work by targeting a mutant kinase mutation having an effect on signaling molecules. These pathways can be modulated by loss of negative regulators leading to kinase activation. Other diseases and disorders including inflammatory disorders and autoimmune disorders can also be identified and therapies targeted appropriately.

As noted above, while researchers have identified a variety of genes and pathways involved in pathologies such as cancer, there is need in the art for additional tools to facilitate the analyses of the regulatory processes that are involved in dysregulated cell growth. Moreover, an understanding of how the products of genes involved in dysregulated cell growth interact in a larger context is needed for the development of improved diagnostic and therapeutic methods for identifying and treating pathological syndromes associated with growth dysregulation. In particular, there remains a need to identify signal transduction events driving oncogenesis and to identify markers useful for assessing progression or inhibition of the oncogenic phenotype.

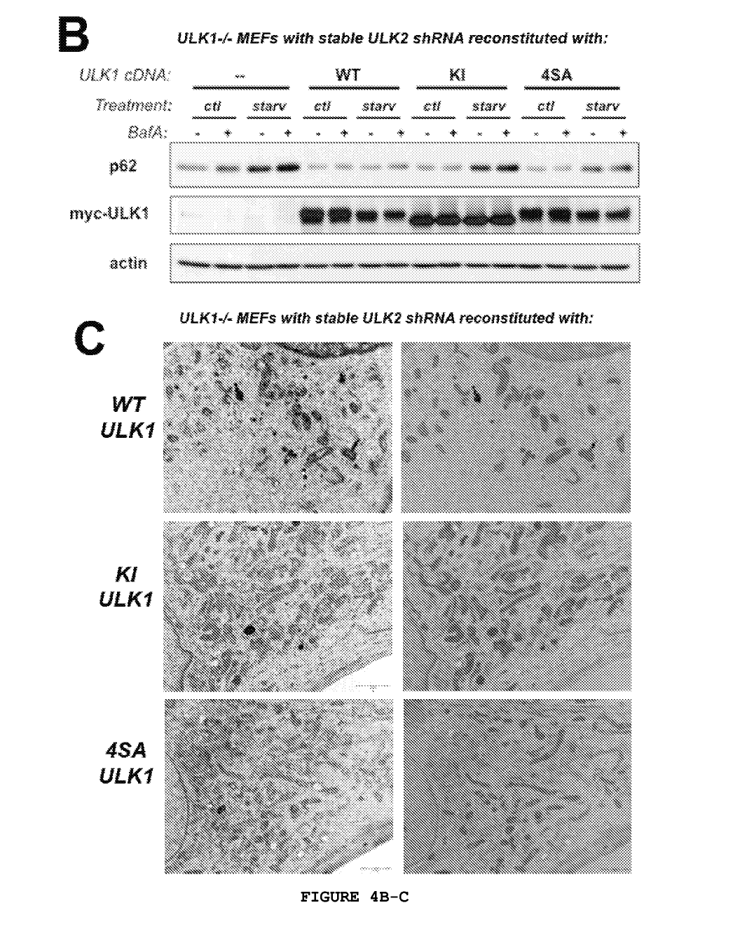

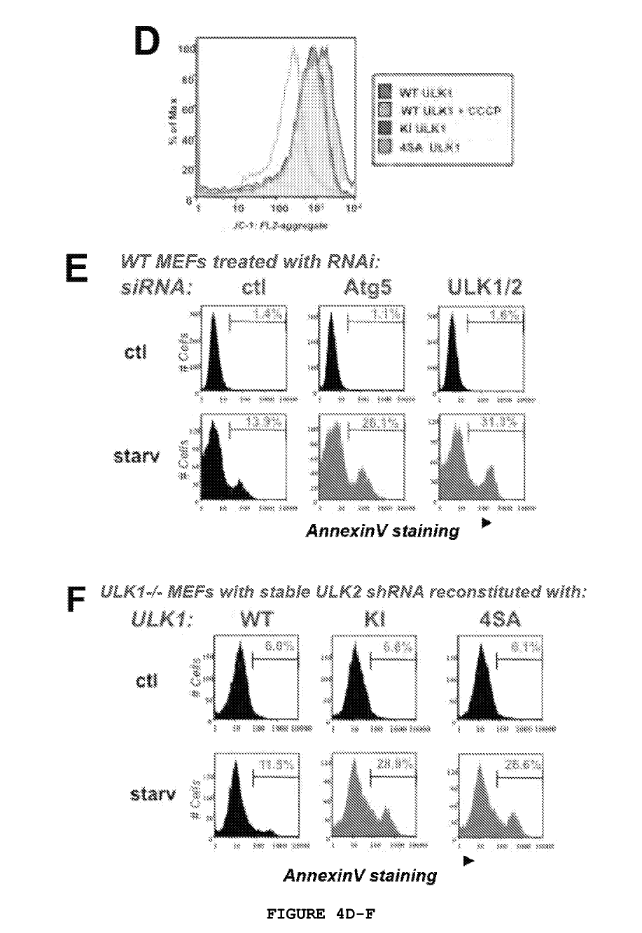

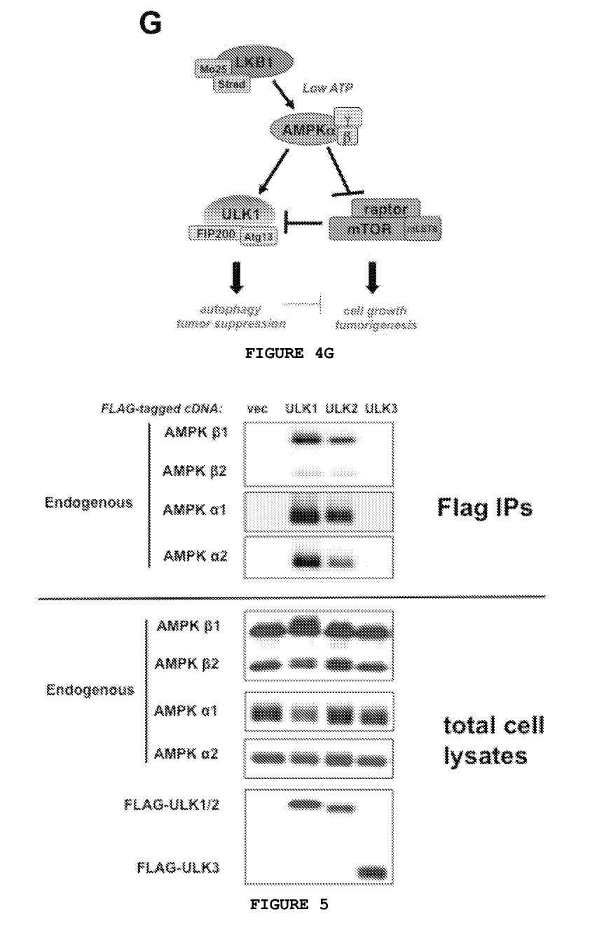

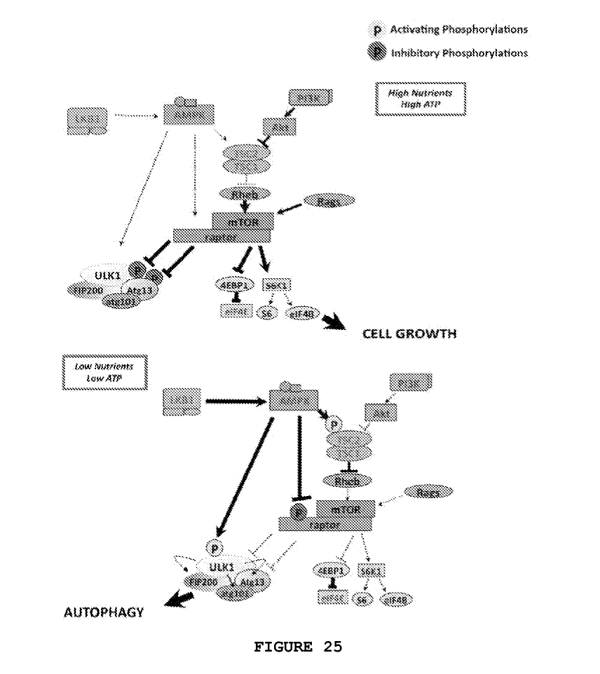

AMP-activated protein kinase (AMPK) and AMPK kinase (AMPKK) comprise a protein kinase cascade (see, e.g., FIGS. 4 and 25). The AMPK cascade regulates fuel production and utilization intracellularly along with a number of other cellular metabolic activities. For example, low cellular fuel (e.g., an increase in AMP concentration) increase AMPK activity. Once activated, AMPK functions either to conserve ATP or to promote alternative methods of ATP generation.

As a highly conserved sensor of cellular nutrient status found in all eukaryotes AMPK is activated in response to lowered intracellular ATP levels, due to nutrient loss or a variety of cellular stresses. Upon energy stress, AMPK serves as metabolic checkpoint, restoring ATP levels through acute regulation of metabolic enzymes and inhibition of pro-growth anabolic pathways. Inactivation of LKB1, the upstream kinase necessary for activation of AMPK under low energy conditions, is a frequent event in several sporadic and inherited forms of human cancer. In addition, LKB1/AMPK signaling is required in liver for the therapeutic effect of metformin, the most prevalent type 2 diabetes drug worldwide, and LKB1 inactivation in murine liver results in a type 2 diabetes like metabolic disease. Thus the LKB1-AMPK pathway provides a direct link between tumor suppression and control of cellular and organismal metabolism.

Similar to AMPK activation, the cellular process of autophagy is also initiated under nutrient poor and low energy states as a survival mechanism to ensure levels of critical metabolic intermediates and to rid the cell of damaged organelles including mitochondria. Genetic studies from budding yeast first delineated the core components of autophagy (ATG genes), and revealed that the most upstream component responsible for autophagy initiation is a conserved kinase complex containing the serine/threonine kinase Atg1 and its associated subunits, Atg13 and Atg17. In mammals this complex is encoded by two Atg1 homologs, ULK1 and ULK2, and the respective subunits Atg13 and FIP200, which signal to the Vps34-Beclin PI3K complex to mediate nucleation of autophagosomal membranes. In yeast and mammalian cells the only characterized signal controlling Atg1/Ulk1 activity is the key metabolic regulator TOR (target of rapamycin) complex 1, which suppresses Atg1 activity under nutrient rich conditions. However to date, no biochemical events that activate Atg1/ULK1/2 have yet been identified.

In order to more fully understand the metabolic pathway regulated and diagnosed in the present disclosure it is useful to understand the components of the pathways involved:

5′AMP-activated protein kinase or AMPK consists of three proteins (subunits) that together make a functional enzyme that plays a role in cellular energy homeostasis. It is expressed in a number of tissues, including the liver, brain, and skeletal muscle. Activation of AMPK has been shown to activate hepatic fatty acid oxidation and ketogenesis, inhibit cholesterol synthesis, lipogenesis, and triglyceride synthesis, inhibit adipocyte lipolysis and lipogenesis, stimulate skeletal muscle fatty acid oxidation and muscle glucose uptake, and modulate insulin secretion by pancreatic beta-cells.

Mammalian AMP-activated kinase (AMPK) is a heterotrimeric protein composed of 1 alpha subunit, 1 beta subunit, and 1 gamma subunit. There are, at least, two known isoforms of the alpha subunit (a1 and a2). AMPKα1 and AMPKα2 have 90% amino acid sequence identity within their catalytic cores but only 61% in their C-terminal tails (see Online Mendelian Inheritance in Man (OMIM) Database Accession No. 602739; publicly available at the following website: ncbi.nlm.nih.gov/entrez/dispomim.cgi?id=602739).

An AMPK (such as AMPKα1 and/or AMPKα2) polypeptide is any known AMPK protein or subunit thereof (such as AMPKα1 and/or AMPKα2). The amino acid sequences of prototypical AMPK subunits (such as AMPKα1 and/or AMPKα2) (and nucleic acids sequences encoding prototypical AMPK subunits (such as AMPKα1 and/or AMPKα2)) are well known. Exemplary AMPKα1 amino acid sequences and the corresponding nucleic acid sequences are described, for instance, in GenBank Accession Nos. NM_{—}206907.3 (GI:94557298)(Homo sapiens transcript variant 2 REFSEQ including amino acid and nucleic acid sequences); NM_{—}006251.5 (GI:94557300)(Homo sapiens transcript variant 1 REFSEQ including amino acid and nucleic acid sequences); NM_{—}001013367.3 (GI:94681060)(Mus musculus REFSEQ including amino acid and nucleic acid sequences); NMJ) 01039603.1 (GI:88853844)(Gallus gallus REFSEQ including amino acid and nucleic acid sequences); and NM_{—}019142.1 (GI: 11862979XRaJfWS norvegicus REFSEQ including amino acid and nucleic acid sequences). Exemplary AMPKα2 amino acid sequences and the corresponding nucleic acid sequences are described, for instance, in GenBank Accession Nos. NM_{—}006252.2 (GI:46877067)(Homo sapiens REFSEQ including amino acid and nucleic acid sequences); NM_{—}178143.1 (GI:54792085) (Mus musculus REFSEQ including amino acid and nucleic acid sequences); NM_{—}001039605.1 (GI:88853850)(Gallus gallus REFSEQ including amino acid and nucleic acid sequences); and NM_{—}214266.1 (GI:47523597) (Mus musculus REFSEQ including amino acid and nucleic acid sequences).

Triggering the activation of AMPK can be carried out with increasing concentrations of AMP. The γ subunit of AMPK undergoes a conformational change at increased concentrations of AMPK so as to expose the active site (Thr-172) on the α subunit. Increased concentrations of AMP will give rise to the conformational change on the γ subunit of AMPK as two AMP bind the two Bateman domains located on that subunit.

Because of the large and diverse cascades involved in AMPK activity, general methods of activation and inhibition have diverse effects some beneficial in some indication and harmful in others. For example, AMPK activation leads to beneficial phosphorylating events downstream as well as negative phosphorylation events downstream. Accordingly, selective regulation of downstream kinases is important in certain disease and disorders.

Macroautophagy (“autophagy”) is a major degradation system, by which cytoplasmic contents are degraded in the lysosomes of cells. An isolation membrane, also known as a phagophore, sequesters a portion of the cytoplasm, which results in the formation of an autophagosome. This autophagosome subsequently fuses with a lysosome, where cytoplasm-derived materials are degraded by lysosomal hydrolases. Resultant amino acids are delivered back to the cytoplasm and then reused or further metabolized. Autophagy is basically a nonselective process, although several proteins are selectively degraded by this pathway. Autophagy is highly conserved among eukaryotes and usually activated by nutrient starvation to produce necessary amino acids within cells, thus helping them adapt to starvation conditions. Autophagy is also important for intracellular protein quality control, preimplantation development, degradation of intracellular pathogens, antigen presentation, tumor suppression, and certain types of cell death.

ULK1, a mammalian serine/threonine protein kinase, plays a role in the initial stages of autophagy. Quantification of the number of cells undergoing cell death following starvation (EBSS) as denoted by Annexin V-positivity revealed that cells expressing kinase-inactive and the 4SA non-phosphorylatable ULK1 underwent significantly more cell death than their wild-type ULK1 expressing counterparts (FIG. 4D). Taken altogether, these findings strongly suggest that AMPK phosphorylation of ULK1 is required for ULK1 function in autophagy and to promote cell survival under conditions of nutrient deprivation.

The polynucleotide and polypeptide sequences of a mouse ULK1 are provided herein below as SEQ ID NO:1 and 2 and human in SEQ ID NO:11 and 12. Alignment of SEQ ID NO:2 and 12 can be performed to identify those sequence of SEQ ID NO:3, 4, 5, and 6 that overlap between human and mouse. For example, SER555 in mouse corresponds to SER556 in humans. Sequences that have 60% amino acid sequence identity with a prototypical ULK1 polypeptide of SEQ ID NO:2 and 12 are also useful; for example, at least 75%, at least 80%, at least 85%, at least 90%, at least 95%, or at least 98% amino acid sequence identity with an amino acid sequence as set forth in SEQ ID NO:2. In other method embodiments, a homolog or functional variant of an ULK1 has one or more conservative amino acid substitutions as compared to a prototypical ULK1 polypeptide; for example, no more than 3, 5, 10, 15, 20, 25, 30, 40, or 50 conservative amino acid changes compared to an amino acid sequence of SEQ ID NO:2 are also useful.

AMPK is an upstream regulator of autophagy. The disclosure demonstrates the process is modulated by ULK1 downstream of AMPK. The disclosure demonstrates a direct connection between energy sensing and core conserved autophagy proteins. Phosphorylation of ULK1 by AMPK is required for ULK1-mediated control of autophagy, demonstrating a direct and causal link between energy sensing and nutrient levels and the first committed step of autophagy. While it remains possible that AMPK may target additional components of the autophagy cascade, the reported control of ULK1 by mTORC1 and the known control of mTORC1 by AMPK allows for a two-pronged mechanism to ensure full activation of ULK1 and commitment to autophagy only under the appropriate cellular conditions. Studies across a number of species suggest that TORC1-dependent signals suppress Atg1/ULK1 through direct phosphorylation of Atg13 and perhaps Atg1/ULK1. As AMPK directly phosphorylates raptor and TSC2 to shut off mTORC1, this suggests that AMPK activation will simultaneously directly phosphorylate residues in ULK1 required for its activation, while suppressing inhibitory mTOR phosphorylation events on distinct ULK1 residues as well as on Atg13 (FIG. 25, FIG. 4E). The fact that AMPK directly phosphorylates both raptor and ULK1, which also associate with one another (FIG. 4E), suggests that localized pools of AMPK may simultaneously inhibit mTORC1 function and promote ULK1 function at a particular endosomal/lysosomal membrane. The spatial coordination of two critical conserved substrates of AMPK affords a direct mechanism to rapidly govern the switch within a cell from catabolism to anabolism.

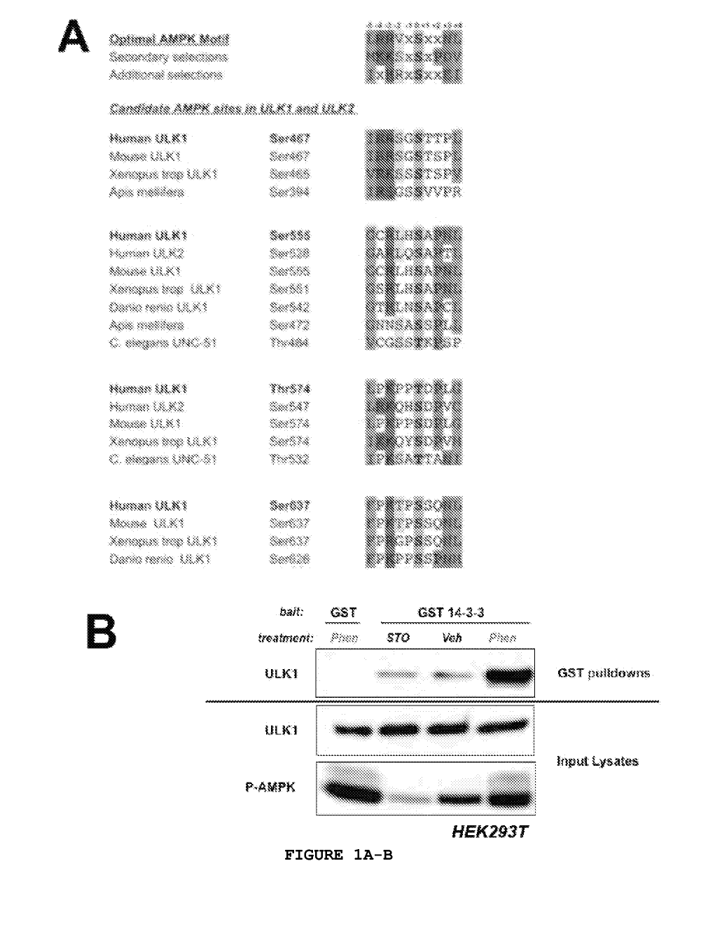

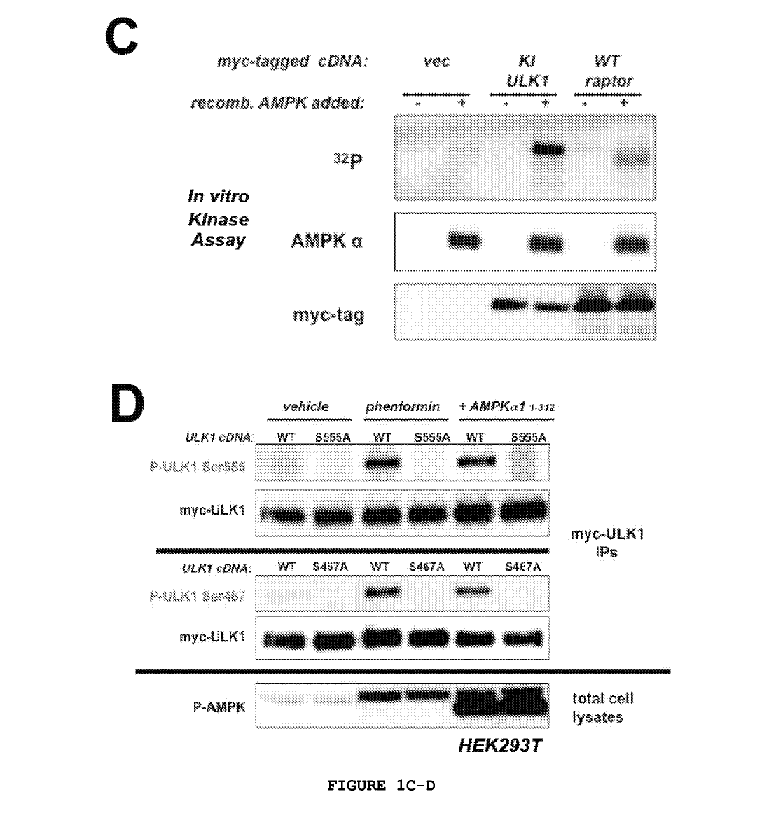

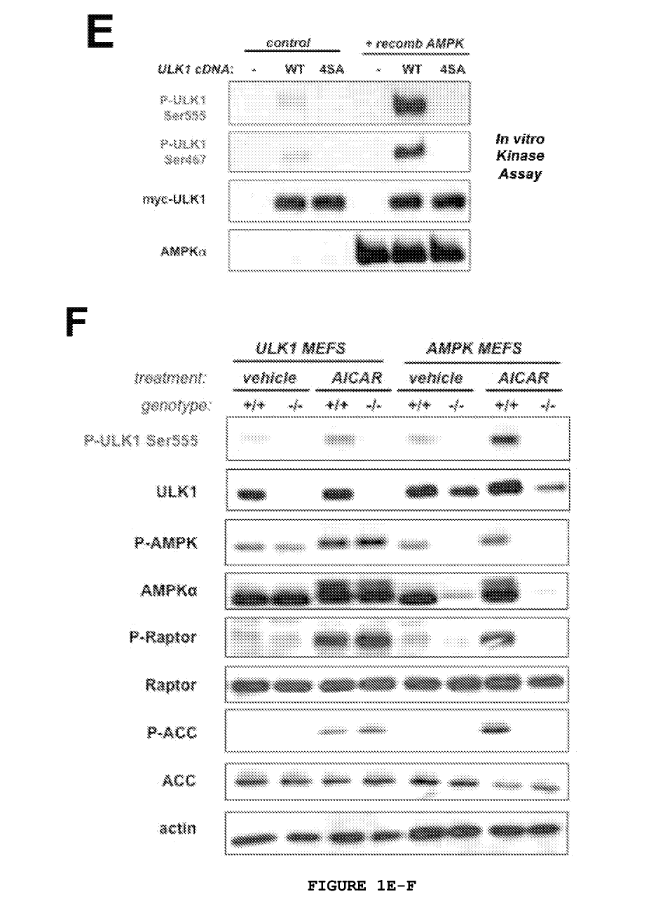

The requisite co-localization of these three kinase complexes also affords a mechanism to rapidly feedback and restore homeostatic control when nutrients become plentiful again. Supporting this concept, genetic evidence in Drosophila demonstrates that ULK1 activity has a feedback to control mTORC1 activity. It also remains possible that ULK1 feeds back to modulate AMPK activity, as hinted at from the reduced AMPK activity apparent here in ULK1-deficient cells (FIG. 1F,G). The disclosure demonstrates that AMPK phosphorylation of ULK1 serves to stimulate autophagy and as such suggests that phosphorylation of ULK1 on Ser467 and Ser555 may serve as some of the earliest markers of autophagy initiation.

Accordingly, modulation of ULK1 phosphorylation can promote cell death and apoptosis. Thus, modulating phosphorylation of ULK1 can provide an effective mechanism for treating cancer and inflammatory diseases and disorders by promoting apoptosis particularly in rapidly growing cells or inhibiting autophagy in cells undergoing induced cellular stress.

Proteins, polypeptides, peptidomimetics and small molecules that regulate autophagy in cancer cells make attractive therapeutic and diagnostic targets. Tumor cells have been observed to exhibit lower levels of autophagic activity. A number of well-known oncogenes and tumor suppressor genes recalibrate autophagic pathways, and thereby alter prospects for cell survival and proliferation. The PTEN tumor suppressor gene, class I PI 3-kinase and Akt oncogenes, Ras and Myc oncogenes are among the proteins that appear to act in this way.

In addition, during cancer therapies cancer cells rely on autophagy in order to evade anti-cancer treatments designed to reduce nutrient supply and enhance the stress on rapidly dividing cells. A compound that downregulates autophagy is a useful additional drug in cancer treatment. The targeting of specific autophagy regulatory proteins rather than a targeting of autophagy in general is useful in treating cancer as well as new modes of diagnosing cancer.

Furthermore, the disclosure provides methods of modulating disease and disorders associated with aberrant phosphorylation of ULK1 and 2. For example, alterations in the autophagy degradation pathway have been described in normal brain aging and in age-related neurodegenerative diseases including Alzheimer's and Parkinson's diseases. See Nixon, R. “Autophagy in neurodegenerative disease: friend, foe, or turncoat?” Trends in Neurosciences (2006) 29(9):528-535. An improper clearance of proteins in these diseases may result either from a compromise in the autophagy degradation pathway or induce alterations in this pathway, and may result in neuron dysfunction and neuron loss. The targeting of specific autophagy regulatory proteins rather than a targeting of autophagy in general is useful in treating neurodegenerative diseases as well as new modes of diagnosing neurodegenerative diseases. Thus, in some methods and compositions of the disclosure increasing autophagy activity of ULK1 can provide beneficial results.

Thus the disclosure provides targets for modulation of autophagy and related disease and disorder and diagnostic methods for determining diseases associated with dysregulated phosphorylation of ULK1. As described more fully below antibodies that specifically bind to the phosphorylated and non-phosphorylated sequences having a consensus sequence of LRRVXSXXNL (SEQ ID NO:7), MKKSXSXPDV (SEQ ID NO:8), IXHRXSXXEI (SEQ ID NO:9) or GXRLXSAPXL (SEQ ID NO:10), wherein X is any amino acid can be used. In specific embodiments, antibodies that specifically bind to the phosphorylated and non-phosphorylated sequences selected from the group consisting of: IRRSGSTTPL (SEQ ID NO:3); GCRLHSAPNL (SEQ ID NO:4); LPKPPTDPLG (SEQ ID NO:5) and FPKTPSSQNL (SEQ ID NO:6) are useful in the methods of the disclosure.

In yet other embodiments, a polypeptide containing a sequence as set forth in SEQ ID NO:3, 4, 5, 6, 7, 8, 9 or 10 or any of the sequences in FIG. 1A can be used in various embodiment of the disclosure. For example, the polypeptides will comprise a sequence containing the phosphorylatable site (e.g., a serine or threonine) of SEQ ID NO:3, 4, 5, 6, 7, 8, 9 or 10 capable of being phosphorylated by AMPK. In particularly embodiments, the polypeptides used as therapeutics or as screening agents containing the sequences will lack ULK activity or autophagy activity. In certain embodiments the polypeptides serve as substrates for AMPK.

As described above and herein, cancer cells can avoid cancer therapies that cause metabolic stress to the cancer cell by inducing autophagy. Accordingly, in one embodiment, the disclosure provides methods and compositions useful for inhibiting the autophagy response in cancer cells undergoing cancer treatment. For example, by contacting a cancer cell with an agent the inhibits phosphorylation or ULK1 or 2 at a site containing the sequence of SEQ ID NO:3, 4, 5, 6 or consensus sequences of SEQ ID NO:7, 8, 9 or 10, ULK autophagy activity may be inhibited thereby promoting the cancer cells metabolic stress and apoptosis. Examples of agents that are useful in the methods and composition of the disclosure include polypeptide or peptides that compete with the phosphorylation of ULK1 or 2 by AMPK.

The disclosure provides a method of inhibiting autophagy and disease or disorder cause by activation of ULK1 by contacting a cell or subject with a motif which is the substrate for AMPK and having identity to a sequence in ULK1 polypeptide (e.g., SEQ ID NO:3, 4, 5, 6, 7, 8, 9 or 10. Because ULK1 is needed for autophagy and is activated by AMPK an inhibitor of phosphorylation (e.g., a competitive peptide inhibitor or antibody or small molecule drug) that prevents AMPK from activating ULK1 would be useful in treating ULK1 associated disease and disorders. Examples of ULK1 diseases and disorders include tumor cells, which due to their metabolic requirements rely on autophagy for survival during cancer therapy. Accordingly, inhibiting the activity of ULK1 inhibits tumor growth and promotes apoptosis.

Some method embodiments involve a functional fragment of ULK1 or a subunit thereof including a ULK1 peptide that is a substrate for AMPK. Functional fragments of ULK1 can be any portion of a full-length or intact ULK1 including, e.g., about 20, about 30, about 40, about 50, about 75, about 100, about 150 or about 200 contiguous amino acid residues of same; provided that the fragment serves as a substrate site for AMPK or an AMPK complex.

For example, fragments of ULK1 containing a sequence as set forth in FIG. 1A including, but not limited to, SEQ ID NO:3, 4, 5, or 6 and the consensus sequence of SEQ ID NO:7, 8, 9 or 10 may be used to compete with ULK1 or 2 as an AMPK substrate. In other embodiments, antibodies that bind to and inhibit ULK1 or 2 phosphorylation at a site comprising SEQ ID NO:3, 4, 5, 6, 7, 8, 9 or 10 can be used. In yet a further embodiment, small molecule inhibitors of AMPK activity or ULK1 or 2 autophagy activity can be used. In yet another embodiment, siRNA or other inhibitory nucleic acids may be used in the methods and compositions of the disclosure to downregulate or inhibit expression of ULK1 or 2.

The methods of above can be used in combination with other anti-cancer therapeutics including, but not limited to, classical chemotherapeutic agents, such as steroids, antimetabolites, anthracycline, vinca alkaloids, antibiotics, alkylating agents, epipodophyllotoxin and anti-tumor agents such as neocarzinostatin (NCS), adriamycin and dideoxycytidine; mammalian cell cytotoxins, such as interferon-α (IFN-α), interferon-βγ (IFN-βγ), interleukin-12 (IL-12) and tumor necrosis factor-α (TNF-α); plant-, fungus- and bacteria-derived toxins, such as ribosome inactivating protein, gelonin, α-sarcin, aspergillin, restrictocin, ribonucleases, diphtheria toxin, Pseudomonas exotoxin, bacterial endotoxins, the lipid A moiety of a bacterial endotoxin, ricin A chain, deglycosylated ricin A chain and recombinant ricin A chain; as well as radioisotopes.

In one embodiment, a phosphorylatable polypeptide or peptide of the disclosure containing the sequence of SEQ ID NO:3, 4, 5, or 6 or consensus sequences of SEQ ID NO:7, 8, 9 or 10 may be fused with a protein transduction domain. The fusion of a protein transduction domain (PTD) with phosphorylatable polypeptide of the disclosure is sufficient to cause their transduction into a variety of different cells in a concentration-dependent manner. Moreover, this technique for protein delivery appears to circumvent many problems associated with DNA and drug based techniques.

PTDs are typically cationic in nature. These cationic protein transduction domains track into lipid raft endosomes carrying with them their linked cargo and release their cargo into the cytoplasm by disruption of the endosomal vesicle. Examples of PTDs include AntHD, TAT, VP22, cationic prion protein domains and functional fragments thereof. The disclosure provides methods and compositions that combine the use of PTDs such as TAT and poly-Arg, with a phosphorylatable domain of the disclosure (e.g., “cargo”) domain. These compositions provide methods whereby the phosphorylatable agent can be more effectively taken up by a cell.

In general, the transduction domain of the fusion molecule can be nearly any synthetic or naturally-occurring amino acid sequence that can transduce or assist in the transduction of the fusion molecule. For example, transduction can be achieved in accord with the invention by use of a protein sequence such as an HIV TAT protein or fragment thereof that is covalently linked at the N-terminal or C-terminal end to the phosphorylatable polypeptide or peptide domain. In some embodiments multiple PTDs may be fused to the phosphorylatable polypeptide or peptide.

The type and size of the PTD will be guided by several parameters including the extent of transduction desired. PTDs will be capable of transducing at least about 20%, 25%, 50%, 75%, 80%, 9 or 100% of the cells of interest, more preferably at least about 95%, 98% and up to, and including, about 100% of the cells. Transduction efficiency, typically expressed as the percentage of transduced cells, can be determined by several conventional methods.

In another embodiment, a phosphorylatable polypeptide or peptide of the disclosure containing the sequence of SEQ ID NO:3, 4, 5, or 6 or the consensus sequence of SEQ ID NO:7, 8, 9 or 10 may be fused to a ligand domain (e.g., a targeting molecule). For example, a ligand domain includes, but is not limited to, a ligand or an antibody that specifically binds to its corresponding target, for example, a receptor on a cell surface. Thus, for example, where the ligand domain is an antibody, the fusion polypeptide will specifically bind (target) cells and tissues bearing the epitope to which the antibody is directed. Thus, a ligand refers generally to all molecules capable of reacting with or otherwise recognizing or binding to a receptor or polypeptide on a target cell. Any known ligand or targeting molecule can be used as the ligand domain of the fusion polypeptide of the invention. Examples of targeting peptides that can be manipulated and cloned or linked to produce a fusion polypeptide are ample in the literature. In general, any peptide ligand can be used or fragments thereof based on the receptor-binding sequence of the ligand. In immunology, such a peptide domain is referred to as an epitope, and the term epitope may be used herein to refer to a ligand recognized by a receptor. For example, a ligand comprises the sequence of a protein or peptide that is recognized by a binding partner on the surface of a target cell, which for the sake of convenience is termed a receptor. However, it should be understood that for purposes of the invention, the term “receptor” encompasses signal-transducing receptors (e.g., receptors for hormones, steroids, cytokines, insulin, and other growth factors), recognition molecules (e.g., MHC molecules, B- or T-cell receptors), nutrient uptake receptors (such as transferrin receptor), lectins, ion channels, adhesion molecules, extracellular matrix binding proteins, and the like that are located and accessible at the surface of the target cell.

The size of the ligand domain peptide can vary within certain parameters. Examples of ligands include, but are not limited to, antibodies, lymphokines, cytokines, receptor proteins such as CD4 and CD8, hormones, growth factors, and the like which specifically bind desired target cells. For example, several human malignancies overexpress specific receptors, including HER2, LHRH and CXCR4. Accordingly, ligands to these receptors can be used in the fusion polypeptides, methods and compositions of the disclosure. Receptor ligand domains are known in the art.

Moreover, while the disclosure focuses largely on the cell biology of AMPK and ULK1 function, there are a number of physiological and pathological contexts where this is likely to play a critical role. Beyond the conserved nature of these signaling events and the role of some autophagy genes as tumor suppressors, AMPK itself is dysregulated in a variety of human cancers and inflammatory disease and disorders bearing inactivating mutations in its upstream kinase LKB1. Thus ULK1 may play a central role in the beneficial effects of the LKB1/AMPK pathway on tumor suppression and treatment of metabolic disease, as observed here with metformin stimulation of ULK1 phosphorylation in liver and the profound defect in autophagy in AMPK-deficient livers.

Thus, in other embodiments, it is beneficial to promote autophagy activity. For example, in protein misfolding or aggregation diseases and disorders of the brain increased autophagy can promote clearing of the misfolded proteins or aggregated proteins from the cytoplasm of the cell. Promoting autophagy in this context can assist in clearing misfolded or aggregate proteins causing a disease or disorder. Thus, in certain embodiments, the disclosure provides methods and compositions that promote autophagy.

In one embodiment, stimulating phosphorylation of ULK1 or 2 is provided to treat diseases and disorders of the brain including, but not limited to, protein misfolding or aggregation disease and disorders selected from the group consisting of Alzheimer disease, Parkinson disease, tauopathies, and polyQ3 expansion diseases. In these embodiments, gene delivery of a functional ULK1 or 2, or functional AMPK can be delivered to the subject or upregulating expression of an endogenous AMPK or ULK to promote autophagy. In other embodiments, small molecule agents such as metformin and derivatives thereof (e.g., phenformin, buformin and the like) can be administered to a subject to promote AMPK activity and ULK phosphorylation to promote autophagy.

In other embodiments, antibodies the specifically bind to a sequence of SEQ ID NO:3, 4, 5, 6, 7, 8, 9 or 10 can be used to modulate or determine phosphorylation of a ULK or homolog thereof. In yet other embodiments, pharmaceutical compositions of the disclosure include agents that promote phosphorylation of a sequence set forth in SEQ ID NO:3, 4, 5, 6, 7, 8, 9 or 10, wherein the phosphorylation increases autophagy in a cell.

Antibodies may be used in the diagnosis of diseases and disorders associated with autophagy.

An “isolated” biological component (such as a polynucleotide, polypeptide, or cell) has been purified away from other biological components in a mixed sample (such as a cell or tissue extract). For example, an “isolated” polypeptide or polynucleotide is a polypeptide or polynucleotide that has been separated from the other components of a cell in which the polypeptide or polynucleotide was present (such as an expression host cell for a recombinant polypeptide or polynucleotide).

The term “purified” refers to the removal of one or more extraneous components from a sample. For example, where recombinant polypeptides are expressed in host cells, the polypeptides are purified by, for example, the removal of host cell proteins thereby increasing the percent of recombinant polypeptides in the sample. Similarly, where a recombinant polynucleotide is present in host cells, the polynucleotide is purified by, for example, the removal of host cell polynucleotides thereby increasing the percent of recombinant polynucleotide in the sample.

Isolated polypeptides or nucleic acid molecules, typically, comprise at least 50%, at least 60%, at least 70%, at least 80%, at least 90%, at least 95% or even over 99% (w/w or w/v) of a sample.

Polypeptides and nucleic acid molecules are isolated by methods commonly known in the art and as described herein. Purity of polypeptides or nucleic acid molecules may be determined by a number of well-known methods, such as polyacrylamide gel electrophoresis for polypeptides, or agarose gel electrophoresis for nucleic acid molecules.

The similarity between two nucleic acid sequences or between two amino acid sequences is expressed in terms of the level of sequence identity shared between the sequences. Sequence identity is typically expressed in terms of percentage identity; the higher the percentage, the more similar the two sequences.

Methods for aligning sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. ScL USA 85:2444, 1988; Higgins and Sharp, Gene 73:237-244, 1988; Higgins and Sharp, CABIOS 5:151-153, 1989; Corpet et al., Nucleic Acids Research 16:10881-10890, 1988; Huang, et al., Computer Applications in the Biosciences 8:155-165, 1992; Pearson et al., Methods in Molecular Biology 24:307-331, 1994; Tatiana et al., (1999), FEMS Microbiol. Lett., 174:247-250, 1999. Altschul et al. present a detailed consideration of sequence alignment methods and homology calculations (J. Mol. Biol. 215:403-410, 1990). The National Center for Biotechnology Information (NCBI) Basic Local Alignment Search Tool (BLAST™, Altschul et al., J. Mol. Biol. 215:403-410, 1990) is available from several sources, including the National Center for Biotechnology Information (NCBI, Bethesda, Md.) and on the Internet, for use in connection with the sequence-analysis programs blastp, blastn, blastx, tblastn and tblastx. A description of how to determine sequence identity using this program is available on the internet under the help section for BLAST™.

For comparisons of amino acid sequences of greater than about 30 amino acids, the “Blast 2 sequences” function of the BLAST™ (Blastp) program is employed using the default BLOSUM62 matrix set to default parameters (cost to open a gap [default=5]; cost to extend a gap [default=2]; penalty for a mismatch [default=−3]; reward for a match [default=1]; expectation value (E) [default=10.0]; word size [default=3]; number of one-line descriptions (V) [default=100]; number of alignments to show (B) [default=100]). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method.

For comparisons of nucleic acid sequences, the “Blast 2 sequences” function of the BLAST™ (Blastn) program is employed using the default BLOSUM62 matrix set to default parameters (cost to open a gap [default=11]; cost to extend a gap [default=1]; expectation value (E) [default=10.0]; word size [default=11]; number of one-line descriptions (V) [default=100]; number of alignments to show (B) [default=100]). Nucleic acid sequences with even greater similarity to the reference sequences will show increasing percentage identities when assessed by this method.

Specific binding refers to the particular interaction between one binding partner (such as a binding agent) and another binding partner (such as a target). Such interaction is mediated by one or, typically, more noncovalent bonds between the binding partners (or, often, between a specific region or portion of each binding partner). In contrast to non-specific binding sites, specific binding sites are saturable. Accordingly, one exemplary way to characterize specific binding is by a specific binding curve. A specific binding curve shows, for example, the amount of one binding partner (the first binding partner) bound to a fixed amount of the other binding partner as a function of the first binding partner concentration. As the first binding partner concentration increases under these conditions, the amount of the first binding partner bound will saturate. In another contrast to non-specific binding sites, specific binding partners involved in a direct association with each other (e.g., a protein-protein interaction) can be competitively removed (or displaced) from such association (e.g., protein complex) by excess amounts of either specific binding partner. Such competition assays (or displacement assays) are very well known in the art.

A cell proliferative disease or disorder refers generally to cells that have an aberrant growth compared to normal cells. Examples of cells comprising a cell proliferative disease or disorder include neoplastic cells and cancer cells. The terms “cancer”, “cancerous”, or “malignant” refer to or describe a disease or disorder characterized by unregulated cell growth. Examples of cancer include but are not limited to astrocytoma, blastoma, carcinoma, glioblastoma, leukemia, lymphoma and sarcoma. More particular examples of such cancers include adrenal, and ophthalmologic cancers, brain cancer breast cancer, ovarian cancer, colon cancer, colotectal cancer, rectal cancer, squamous cell cancer, small-cell lung cancer, non-small cell lung cancer, Hodgkin's and non-Hodgkin's lymphoma, testicular cancer, esophageal cancer, gastrointestinal cancer, renal cancer, pancreatic cancer, glioblastoma, cervical cancer, glioma, liver cancer, bladder cancer, hepatoma, endometrial carcinoma, salivary gland carcinoma, kidney cancer, liver cancer, prostate cancer, vulval cancer, thyroid cancer, hepatic carcinoma and various types of head and neck cancer.

In some embodiments, the disclosure provides for a pharmaceutical composition comprising a pharmaceutically acceptable carrier, an excipient, and an isolated polypeptide or peptide comprising a phosphoryatable site capable of being phosphorylated by AMPK and which modulated autophagy. In one embodiment, the polypeptide or peptide comprises a sequence that is substantially identical to a ULK1 peptide of about 6-100 amino acids, 6-50 amino acids, 6 to 20 amino acids, 10-100 amino acids, 10-50 amino acids, 10-20 amino acids (e.g., 6, 7, 8, 9, 10, 11, 12, 13, 14, 15, 16 or more amino acids) and contains a sequence set forth in SEQ ID NO:3, 4, 5, or 6 or the consensus sequence of SEQ ID NO:7, 8, 9 or 10. The pharmaceutical compositions can be used to modulate the rate of autophagy in vertebrate animals including mammals. Alternatively, the pharmaceutical compositions can be used to inhibit or reduce the rate of autophagy in vertebrate animals, including mammals.

Accordingly, the compositions are considered useful for treating or preventing a variety of conditions including ischemic brain injury, Alzheimer's disease, Parkinson's disease, amyotrophic lateral sclerosis, prion diseases and polyglutamine disorders including Huntington's disease and various spinocerebellar ataxias, transmissible spongiform encephalopathies such as Creutzfeldt-Jakob disease, breast cancer, ovarian cancer, brain cancer, pancreatic cancer, esophageal cancer, colorectal cancer, liver cancer, prostate cancer, renal cancer, lung cancer, Myocardial ischemia, cardiac remodeling, cardiomyopathy, hemodynamic stress, myocardial hypertrophy, Neuronal ceroid-lipofuscinosis (adult and juvenile), Multiple Sulfatase Deficiency (MSD) and Mucopolysaccharidosis type IIIA, Batten disease, Niemann-Pick C, Danon disease, Pompe disease, and dysfunction of innate and adaptive immunity against intracellular pathogens.

The polypeptide and peptide compounds may be formulated into the compositions as neutral or salt forms. Pharmaceutically acceptable non-toxic salts include the acid addition salts (formed with the free amino groups) and which are formed by reaction with inorganic acids such as, for example, hydrochloric, sulfuric or phosphoric acids, or organic acids such as, for example, acetic, oxalic, tartaric, mandelic, citric, malic, and the like. Salts formed with the free carboxyl groups may be derived from inorganic bases such as, for example, sodium, potassium, ammonium, calcium, or ferric hydroxides, and such organic bases such as amines, i.e., isopropylamine, trimethylamine, 2-ethylamino ethanol, histidine, procaine, and the like.

A polypeptide or peptide of the disclosure is suitably administered to a subject, e.g., a human or a non-human mammal, such as a domestic animal. The amount administered may vary depending on various factors including, but not limited to, the agent chosen, the disease, and whether prevention or treatment is to be achieved. The peptides may be administered locally or systemically. Administration of the therapeutic agents may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses.

One or more suitable unit dosage forms comprising a polypeptide or peptide can be administered by a variety of routes including oral, or parenteral, including by rectal, buccal, vaginal and sublingual, transdermal, subcutaneous, intravenous, intramuscular, intraperitoneal, intrathoracic, intracoronary, intrapulmonary and intranasal routes. The dosage form may optionally be formulated for sustained release. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

When the polypeptide or peptide of the disclosure is prepared for oral administration, it is preferably combined with a pharmaceutically acceptable carrier, diluent or excipient to form a pharmaceutical formulation, or unit dosage form. The total active ingredients in such formulations comprise from 0.1 to 99.9% by weight of the formulation. By “pharmaceutically acceptable” it is meant the carrier, diluent, excipient, and/or salt must be compatible with the other ingredients of the formulation, and not deleterious to the recipient thereof. The active ingredient for oral administration may be present as a powder or as granules; as a solution, a suspension or an emulsion; or in achievable base such as a synthetic resin for ingestion of the active ingredients from a chewing gum. The active ingredient may also be presented as a bolus, electuary or paste. A useful reference describing pharmaceutically acceptable carriers, diluents and excipients is Remington's Pharmaceutical Sciences (Mack Publishing Co. N.J. USA, 1991) which is incorporated herein by reference. Supplementary active compounds can also be incorporated into the compositions.

Pharmaceutical formulations containing a polypeptide or peptide of the disclosure can be prepared by procedures known in the art using well known and readily available ingredients. For example, the natriuretic peptide can be formulated with common excipients, diluents, or carriers, and formed into tablets, capsules, suspensions, powders, and the like. Examples of excipients, diluents, and carriers that are suitable for such formulations include the following fillers and extenders such as starch, sugars, mannitol, and silicic derivatives; binding agents such as carboxymethyl cellulose, HPMC and other cellulose derivatives, alginates, gelatin, and polyvinyl-pyrrolidone; moisturizing agents such as glycerol; disintegrating agents such as calcium carbonate and sodium bicarbonate; agents for retarding dissolution such as paraffin; resorption accelerators such as quaternary ammonium compounds; surface active agents such as cetyl alcohol, glycerol monostearate; adsorptive carriers such as kaolin and bentonite; and lubricants such as talc, calcium and magnesium stearate, and solid polyethyl glycols.

For example, tablets or caplets containing the nucleic acid molecule or peptide of the invention can include buffering agents such as calcium carbonate, magnesium oxide and magnesium carbonate. Caplets and tablets can also include inactive ingredients such as cellulose, pregelatinized starch, silicon dioxide, hydroxy propyl methyl cellulose, magnesium stearate, microcrystalline cellulose, starch, talc, titanium dioxide, benzoic acid, citric acid, corn starch, mineral oil, polypropylene glycol, sodium phosphate, and zinc stearate, and the like. Hard or soft gelatin capsules containing the nucleic acid molecule or peptide of the invention can contain inactive ingredients such as gelatin, microcrystalline cellulose, sodium lauryl sulfate, starch, talc, and titanium dioxide, and the like, as well as liquid vehicles such as polyethylene glycols (PEGs) and vegetable oil. Moreover, enteric coated caplets or tablets of the nucleic acid molecule or peptides of the disclosure are designed to resist disintegration in the stomach and dissolve in the more neutral to alkaline environment of the duodenum.

The ULK1 peptide may be prepared in an injectable formulation. Injectable preparations, for example sterile injectable aqueous or oleaginous suspensions, are formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid find use in the preparation of injectables.

A polypeptide comprising a sequence of SEQ ID NO:3, 4, 5, 6, 7, 8, 9 or 10 may be used as an immunogen, with an exemplary use being to generate antibodies. The polypeptide or peptide may comprise a phosphoserine or serine or threonine or phosphothreonine. In some embodiments, the isolated polypeptide or peptide of the above embodiments may be conjugated to a carrier to enhance the peptide's immunogenicity. Use of carriers while immunizing the animal is preferred when producing antibodies against a polypeptide or peptide. It is generally appreciated in the art that antigens must be at least 10 kDa in order to elicit a satisfactory immune response. The size of an antigen can be effectively increased by association of the antigen with a carrier.

The carrier may be a carrier protein. Alternatively, the ULK1 peptide and the carrier protein may be part of a fusion protein. Exemplary carriers include, but are not limited to, Keyhole limpet cyanin, BSA, cationized BSA, ovalbumin, blue carrier immunogenic protein, avidin, BTG, bovine G globulin, bovine Immunoglobulin G (BlgG), bovine thyroglobulin, conalbumin, colloidal gold, edestin, exoprotein A (recombinant) from P. aeruginosa, hemocyanin from crab P. camtschatica, Helix promatia Hemocyanin (HPH), HSA, KTI (Kuntz trypsin inhibitor from soybeans), LPH (Heamocyanin from Limulus polyphemus), Pam3Cys-Th, polylysine, porcine thyroglobulin (PTG), purified protein derivative (PPD), rabbit serum albumin (RSA), soybean trypsin inhibitor (STI), sunflower globulin (SFG), and Tetanus toxoid. The carrier protein may be coupled to the peptide as described in Lateef, S. et al J. Biomol. Tech. (2007) 8:173-176.

The following examples are intended to illustrate but not limit the disclosure.

### Example

Plasmids

The cDNA encoding human Atg13 (KIAA0652/AB014552) was obtained from Kazusa DNA Research Institute in Japan. The cDNAs for human FIP200, mouse ULK1, and mouse ULK2 constructs were obtained from Open Biosystems (clones 3908134, 6834534, and 5709559 respectively). Human Atg101 and human ULK3 isoform 2 was obtained from Invitrogen (clones 60673 and 10H45122 respectively). The myc tag and attL1 sites (for BP reaction) were added by PCR to the N-terminus of ULK1 using the standard procedure. cDNAs were subcloned into pDONR221 with BP clonase (Invitrogen), and site-directed mutagenesis was performed using QuikChange II XL (Stratagene) according to the manufacturers instructions. Kinase dead ULK1 was achieved by a K46I mutation. Wild type and mutant alleles in pDONR221 were sequenced in their entirety to verify no additional mutations were introduced during PCR or mutagenesis steps and then put into either mammalian expression pDest15 GST bacterial expression vector, pcDNA3 myc mammalian expression vector, or pcDNA6.2 V5 dest (Invitrogen), or pQCXIN retroviral destination vector (Addgene 17399) by LR reaction (Invitrogen). pEBG-AMPK α1 (1-312) is constitutively active by truncation after amino acid 312. pEBG and pEBG-14-3-3 constructs and human myc-Raptor onstructs available from Addgene (plasmids#22227, 1942, 1859, 18118 respectively).

Antibodies and Reagents.

Cell Signaling Antibodies used: pAMPK Thr172 (#2535), AMPK α1 (#2532), AMPK α2 (2757) AMPK beta 1/2 (#4150), AMPK α1/2 (#2532), Atg5 (#2630), pACC Ser79 (#3661), ACC (3662), pRaptor Ser792 (#2083), pULK1 Ser467 (#4634), ubiquitin (#3933), Raptor (#2280), Myc poly (#2278), Myc 9B11 (#2276), COX IV (4844), GST (#2622), LC3B (#3868). Anti-ULK1 (A7481), M2 agarose (A2220) actin (A5441), and Flag poly (F7425) from Sigma. Guinea pig anti-p62 sequestosome antibody from Progen, Heidelberg Germany (03-GPP62-C). TOM20 antibody from Santa Cruz (FL-145). Phospho-ULK1 Ser555 was developed in collaboration with Gary Kasof at Cell Signaling Technology. GSH sepharose from GE Healthcare. Active recombinant AMPK was obtained from Millipore (cat#14-305). AICAR was obtained from Toronto Research Chemicals. Bafilomycin A, Phenformin, and metformin from Sigma and Phenformin and metformin were dissolved in DMEM with 10% FBS. Earle's Buffered Salk Solution (EBSS), Medium 199 (for primary hepatocytes), Protein G sepharose, and Mitotracker Red CMXRos from Invitrogen. A769662 from Abbott Labs. Annexin V-PE Apoptosis Detection Kit from BD Biosciences, and JC-1 dye and CCCP from Molecular Probes. STO-609 from VWR. TPP plates for primary hepatocytes from Light Lab Systems.

shRNA Target Sequences:

TRC lentiviral shRNAs targeting ULK1 or ULK2 were obtained from Sigma.
Human ULK1 shRNA #5: TRCN0000000835
Human ULK1 shRNA #6: TRCN0000000836
Human ULK1 shRNA #7: TRCN0000000837
Human ULK1 shRNA #8: TRCN0000000838
Human ULK1 shRNA #9: TRCN0000000839
Human ULK2 shRNA #89: TRCN0000000889
Human ULK2 shRNA #90: TRCN0000000890
Human ULK2 shRNA #91: TRCN0000000891
Human ULK2 shRNA #92: TRCN0000000892
Human ULK2 shRNA #93: TRCN0000000893
Mouse ULK2 shRNA #20: TRCN0000278670
Mouse ULK2 shRNA #38: TRCN0000278671
Mouse ULK2 shRNA #65: TRCN0000026765
Mouse ULK2 shRNA #93: TRCN0000026693
Mouse ULK2 shRNA #95: TRCN0000026695

Cell Culture and Transfection.

HEK293T, U2OS, and mouse embryonic fibroblast (MEF) cells were cultured in DMEM containing 10% fetal bovine serum (HyClone) and penicillin/streptomycin at 37° C. in 5% CO2. For transient expression of proteins and packaging of virus, HEK-293T cells were transfected with DNA or short hairpin RNA (shRNA) plasmids using Lipofectamine 2000 (Invitrogen) following the manufacturer's protocol. SV40 immortalized wild-type and ULK1 knockout MEFs. Primary ULK1 wt and −/− MEFs were isolated. Briefly, embryos were harvested, the heads and internal organs were removed, and the carcasses were finely minced with scissors and digested by incubation in 0.05% trypsin-0.5 mM EDTA solution for 30 min at 37° C. with gentle agitation. Trypsin was inactivated by adding high-glucose Dulbecco modified Eagle medium (DMEM; Invitrogen, Carlsbad, Calif.) supplemented with 10% heat-inactivated fetal bovine serum (FES; Invitrogen) and antibiotics. Homogeneous cell suspensions were plated in DMEM containing 10% FES and maintained at 37° C. as monolayer cultures. The genotype of each source of primary MEFs was determined by PCR. Primary wild-type and AMPKα1^{-/-}, a2^{-/-} double knockout MEFs.

Primary hepatocytes were isolated. Briefly, livers were perfused with Hank's balanced salt solution (HESS, KCl, 5.4 mM; KH₂PO₄, 0.45 mM NaCl, 138 mM; NaHCO₃,4.2 mM; Na₂HPO₄, 0.34 mM; glucose, 5.5 mM; HEPES, 1 M; EGTA, 50 mM; CaCl₂, 50 mM; pH 7.4). Livers were washed at a rate of 5 ml/min using the portal vein before collagenase (0.025%) was added. Cell viability was assessed by the trypan blue exclusion test and was always higher than 60%. Hepatocytes were seeded at a density of 2×10⁶ cells in medium M199 with Earle salts (Invitrogen), supplemented with 10 g/ml of streptomycin, 100 units/ml of penicillin, and 2.4 mM of glutamine onto TPP plates (Light Lab Systems). After cell attachment (6 h), the medium was replaced by fresh M199 medium for 24 h before indicated treatments.

For RNAi experiments, smartpools from Dharmacon against mouse ULK1, ULK2, or Atg5 (L-040155-00-0005, L-040619-00-0005, and L-064838-00-0005 respectively) were transfected at a final concentration of 20 nM according to the manufacturers instructions using RNAiMAX (Invitrogen). Cells were subsequently placed in EBSS or DMEM with 10% FBS media as a control for the indicated times, and were lysed 72 hours post-transfection. FACS analysis to analyze cell death described below.

Lenti- and Retroviral Preparation and Viral Infection.

Lentiviral shRNA transduction and retroviral gene expression was performed. Briefly, for retroviral infection, the pQCXIN myc ULK1 constructs were transfected along with the ampho packaging plasmid into growing HEK293T cells. Virus containing supernatants were collected 48 hours after transfection, filtered to eliminate cells and target ULK1−/− MEFs or U2OS were infected in the presence of polybrene. 24 hours later, cells were selected with neomycin. The pLKO shRNA vectors encoding shRNAs were transfected into HEK293T cells with lentiviral packaging plasmids vsvg, GAG/pol, and REV using Lipofectamine 2000. Viruses were collected 48 hours after transfection, and MEFs (shRNA #93 against mULK2) and U2OS (shRNA #8 and #91 against hULK1 and hULK2 respectively) already stably expressing myc-ULK1 were infected with the collected viruses for 4 h in the presence of polybrene to knock down the endogenous proteins.

Cell Lysis, Immunoprecipitations, and Mammalian Autophagy Analysis.

Cells were harvested 24 hours after transfection for co-immunoprecipitation assay and Western blot analysis. Cells were rinsed once with ice-cold PBS and lysed in ice-cold lysis buffer (20 mM Tris pH 7.5, 150 mM NaCl, 1 mM EDTA, 1 mM EGTA, 0.3% CHAPS, 2.5 mM pyrophosphate, 50 mM NaF, 5 mM b-glycero-phosphate, 50 nM calyculin A, 1 mM Na₃VO₄, and proteased inhibitors (Roche). The soluble fractions of cell lysates were isolated by centrifugation at 13,000 rpm for 10 minutes. For immunoprecipitations, primary antibodies were added to the lysates and incubated with rotation for 1.5 hours at 4° C. 60 μl of a 50% slurry of protein G-sepharose was then added and the incubation continued for an additional 1 hour. Immunoprecipitates were washed three times with cold lysis buffer before addition of sample buffer. immunoprecipitated proteins were denatured by the addition of 20 μl of sample buffer and boiling for 5 minutes, resolved by 8%-16% SDSPAGE, and analyzed by immunoblotting as described. For analysis of autophagy by western blots, cells were plated at a density of 2.0×10⁵ per dish in 6 cm dishes and grown in DMEM plus 10% FBS, penicillin, and streptomycin. Twenty hours after plating, the growth medium was replaced with aforementioned medium (ctl), or starvation (EBSS) for the indicated times with or without 100 nM bafilomycin A and lysed in boiling lysis buffer (10 mM Tris pH7.5, 100 mM NaCl, 1% SDS). After trituration, lysates were equilibrated for protein levels using the BCA method (Pierce) and resolved on 6 to 12% SDS-PAGE gels, depending on the experiment. Quantification of westerns blots was achieved using Kodak Multi Gage software and normalizing to the appropriate loading control (actin when comparing p62 or LC3, or total protein level when comparing phospho-specific antibodies). In all cases, starvation is EBSS (Earle's Buffered Salt Solution) (Invitrogen). Data shown from all cell experiments is representative of 3 independent experiments.

AMPK and ULK1 Kinase Assays.

Gamma 32P assays to measure ULK1 kinase activity. Briefly, myc ULK1 was transfected into HEK293T cells and 20 hours later treated as indicated. The immunoprecipitate was washed in IP buffer 3 times, and washed in kinase buffer (25 mM MOPS, pH 7.5, 1 mM EGTA, 0.1 mM Na3VO4, 15 mM MgCl₂). ATP was added at a 100 μM final concentration. Reactions were performed for 20 minutes at 30° C. Reactions were boiled, run out on SDS page gel. The gel was dried, and imaged using Phospholmager software. In vitro kinase assays to asses AMPK activity on ULK1 were performed using the same protocol as above, but using 0.1 Upper r×n of partially purified rat liver active AMPK heterotrimer (Millipore cat#14-305) and ˜1 ug of purified myc-tagged ULK1 or ˜2 ug of myc-tagged Raptor in a reaction containing 50 mM Tris pH 7.5 and 10 mM MgC12 at 30° C. for 15 minutes. The units here are 1380 U/mg where one unit of AMPK activity is defined as 1 nmol of phosphate incorporated into 200 uM of the AMARA substrate peptide (AMARAASAAALARRR) per minute at 30 degrees C. with a final ATP concentration of 100 uM. Myc-tagged Raptor or ULK1 was purified from transiently transfected plates of HEK293T cells. The amount of immunoprecipitated Raptor or ULK1 was initially estimated from comparing the colloidal blue stained amount of immunoprecipitated protein per 10 cm plate lysed and compared to BSA standards. Quantification of 32P signal was done using Phospholmager and Kodak Multi Gage software. For cold assays AMPK assays, reactions were run out on SDS page gel, transferred, and blotted with a phospho-specific antibody against either serine-467 or 555 of ULK1.

Fluorescence Microscopy Methods.

MEFs reconstituted with myc ULK1 were plated on glass coverslips at a density of 3×10⁵ cells per well in 6-well tissue culture plates. 18 h later, cells were fixed in 4% PFA in PBS for 10 minutes and permeabilized in 0.2% Triton in PBS for 10 minutes. The following primary antibodies were used: mouse anti-myc epitope and LC3B XP antibody (2276 and 3868 respectively, Cell Signaling Technologies). Secondary antibodies were anti-rabbit Alexa488 and anti-mouse Alexa594 (Molecular Probes, 1:1000). Mitotracker Red CMXRos (Invitrogen) was added to live cells at a concentration of 50 nM for 15 minutes. Coverslips were mounted in FluoromountG (SouthernBiotech). 10 random fields per condition were acquired using the 100× objective and representative images shown. Primary hepatocytes were isolated from ULK1^{-/-}, AMPKα1^{-/-}, a2^{-/-} double knockout, or matched wt littermates and the cells were plated at confluency on TPP plates, then 48 hours washed with PBS and fixed in 4% cold PFA 20 hours later. TOM20 (FL-145) used according to the manufacturers instructions at 1:200 overnight incubation at 4 degrees. Glass coverslips were mounted directly on plate with FluoromountG. All confocal microscopy was performed on an LSM 710 spectral confocal microscope mounted on an inverted Axio Observer Z1 frame (Carl Zeiss, Jena, Germany). LC3 puncta were labeled with anti LC3B (#3868 from Cell Signaling Technology) and counterstained with DAPI. Excitation for both markers was provided by a 405 nm solid-state diode laser (for DAPI) and the 488 nm line of an Argon-ion laser (for green) respectively. Laser light was directed to the sample via two separate dichroic beamsplitters (HFT 405 and HFT 488) through a Plan-Apochromat 63×1.4NA oil immersion objective (Carl Zeiss, Jena Germany). Fluorescence was epi-collected and directed to the detectors via a secondary dichroic mirror. DAPI fluorescence was detected via a photomultiplier tube (PMT) using the spectral window 430-480 nm. Green fluorescence was detected on a second photomultipler tube (PMT) with a detection window of 500-570 nm. Confocal slice thickness was typically kept at 0.8 microns consistently for both fluorescence channels with 10 slices typically being taken to encompass the three-dimensional entirety of the cells in the field of view. Maximum intensity projections of each region were calculated for subsequent quantification and analysis.

Quantifying Endogenous LC3 Puncta.

In order to quantify the endogenous LC3 puncta, which were stained with anti LC3 antibody (Cell Signaling #3868), an ImageJ macro was utilized. Briefly, an individual cell within a field of view was selected via the polygon tool. The RGB image is split into its component red, green and blue channels, with the green channel being extracted to an 8-bit grayscale format. This image was then thresholded to two standard deviations above the background signal. Care was taken to ensure consistency of thresholding over multiple fields of view and samples. Once thresholded, the grayscale image was photographically inverted to black pixels over a white background. Once this process was complete, the analyze particles algorithm within ImageJ was employed to measure the number of puncta within a specified region of interest (i.e. a single cell). This process was completed for at least 6 cells within over 10 separate fields of view for each sample and is representative of 3 independent experiments.

Electron Microscopy.

Primary hepatocytes grown on TPP plates or MEFs grown in 60 mm plastic culture dishes were fixed in 2.5% glutaraldehyde in 0.1M Na cacodylate buffer (pH7.3), washed and fixed in 1% osmium tetroxide in 0.1M Na cacodylate buffer. They were subsequently treated with 0.5% tannic acid followed by 1% sodium sulfate in cacodylate buffer and then dehydrated in graded ethanol series. The cells were transitioned in HPMA (2-hydroxypropyl methacrylate: Ladd Research, Williston Vt.) and embedded in LX112 resin. Following overnight polymerization at 60 degrees C., small pieces of resin were attached to blank blocks using SuperGlue. Thin sections (70 nm) were cut on a Reichert Ultracut E (Leica, Deerfield, Ill.) using a diamond knife (Diatome, Electron Microscopy Sciences, Hatfield Pa.), mounted on parlodion coated, copper, slot grids and stained in uranyl acetate and lead citrate. Sections were examined on a Philips CM100 TEM (FEI, Hillsbrough, Oreg.) and data documented on Kodak SO-163 film for later analysis. Alternatively the samples were documented on an Olympus-SIS Megaview III CCD camera (Lakewood, Colo.). For quantification of mitochondria, TEM micrographs were imported into CRT (Cambridge Research & Instrumentation) image analysis software in Form (version 1.0.0). The intracellular compartments for the nucleus and cytoplasm and the mitochondria were pseudocolored and segmented for quantification. Non-cell-containing image areas were subtracted from the analysis. Calculation for the adjusted total mitochondrial area per cell was as follows: (percent area mitochondria)/((percent area cytoplasm+percent area mitochondria)−(percent area nucleus)). Error bars shown are equal to mean+/− Standard Error of the Mean (SEM) N=10.

Mass Spectrometry.

Myc-ULK1 overexpressed in HEK293T cells was treated with vehicle or 5 mM phenformin for 1 hour, IP'd with anti myc antibody (Cell Signaling), run out on SDS-PAGE gel and coomassie stained. Bands on the gel corresponding to ULK1 were cut out and subjected to reduction with dithiothreitol, alkylation with iodoacetamide, and in-gel digestion with trypsin or chymotrypsin overnight at pH 8.3, followed by reversed-phase microcapillary/tandem mass spectrometry (LC/MS/MS). LC/MS/MS was performed using an Easy-nLC nanoflow HPLC (Proxeon Biosciences) with a self-packed 75 μm id×15 cm C18 column coupled to a LTQ-Orbitrap XL mass spectrometer (Thermo Scientific) in the datadependent acquisition and positive ion mode at 300 mL/min. Peptide ions from AMPK predicted phosphorylation sites were also targeted in MS/MS mode for quantitative analyses. MS/MS spectra collected via collision induced dissociation in the ion trap were searched against the concatenated target and decoy (reversed) single entry ULK1 and full Swiss-Prot protein databases using Sequest (Proteomics Browser Software, Thermo Scientific) with differential modifications for Ser/Thr/Tyr phosphorylation (+79.97) and the sample processing artifacts Met oxidation (+15.99), deamidation of Asn and Gln (+0.984) and Cys alkylation (+57.02). Phosphorylated and unphosphorylated peptide sequences were identified if they initially passed the following Sequest scoring thresholds against the target database: 1+ ions, Xcorr≧2.0 Sf≧0.4, P≧5; 2+ ions, Xcorr≧2.0, Sf≧0.4, ≧P 5; 3+ ions, Xcorr≧2.60, Sf≧0.4, P≧5 against the target protein database. Passing MS/MS spectra were manually inspected to be sure that all band Yfragment ions aligned with the assigned sequence and modification sites. Determination of the exact sites of phosphorylation was aided using FuzzyIons and GraphMod and phosphorylation site maps were created using ProteinReport software (Proteomics Browser Software suite, Thermo Scientific). False discovery rates (FDR) of peptide hits (phosphorylated and unphosphorylated) were estimated below 1.5% based on reversed database hits.

Relative Quantification of Phosphorylation Sites.

For relative quantification of phosphorylated peptide signal levels, an isotope-free (label-free) method was used by first integrating the total ion counts (TIC) for each MS/MS sequencing event during a targeted ion MS/MS (TIMM) experiment or a data dependant acquisition. For each targeted phosphorylation site, a ratio of phosphorylated peptide signal (TIC of phosphorylated form) to the total peptide signal (TIC of phosphorylated form+TIC of non-phosphorylated form) for both the insulin and insulin plus rapamycin treated samples were calculated according to the following equation:

TIC_{PO4}/(TIC_{PO4}+TIC_{nonPO4})=Ratio of phosphopeptide signal(R_{PO4})

These ratios of phosphopeptide signal were then compared to the same phosphopeptide ratios from the unstimulated samples according to the following equation:

[(R_{PO4}Unstimulated/R_{PO4}Stimulated−1]×100=% change in phosphorylation level upon treatment

While a direct comparison of phosphopeptide signals between different experimental conditions is not accurate due to differences in sample content, a comparison of the relative ratios of the phosphorylated to non-phosphorylated peptide forms between samples is an accurate measure of signal-level change since the total peptide signal (modified and unmodified) is measured. The above calculations were performed manually using Microsoft Excel and with automated in-house developed software named Protein Modification Quantifier v1.0 (Beth Israel Deaconess Medical Center, Boston, Mass.).

Mice Strains and Tissue Isolation.

AMPKα1^{-/-} mice and AMPKα2^{lox/lox} were serially crossed onto the FVB strain for 4 generations and then intercrossed to generate AMPKα1^{+/-} AMPKα2^{lox/+} and AMPKα1^{-/-} AMPKα2^{lox/lox} littermates. 8 week old male mice were tail-vein injected with adenovirus bearing Cre-recombinase. Deletion of AMPKα2 was examined by immunoblotting with AMPKalpha antibodies which recognize both AMPKalpha1 and AMPKalpha2. ULK1^{-/-} mice were also crossed onto the FVB background for 3 generations prior to analysis. Experimental mice were cervically dislocated and liver and muscle were harvested immediately and either processed for histological analysis (10% formalin) or frozen in liquid nitrogen for molecular studies. These samples were then placed frozen into Nunc tubes, pulverized in liquid nitrogen, and homogenized in lysis buffer (20 mM Tris pH 7.5, 150 mM NaCl, 1 mM EDTA, 1 mM EGTA, 1% Triton X-100, 2.5 mM pyrophosphate, 50 mM NaF, 5 mM b-glycero-phosphate, 50 nM calyculin A, 1 mM Na3VO4, 10 mM PMSF, 4 μg/ml leupeptin, 4 μg/ml pepstatin, 4 μg/ml aprotinin) on ice for 30s using a tissue homogenizer. Total protein was normalized using BCA protein kit (Pierce) and lysates resolved on SDS-PAGE gel.

Flow Cytometry. Cells were seeded at a concentration of 2.5×105 cells/mL, grown overnight (18 hrs) and treated with Vehicle (DMEM+10% FBS) or EBSS or CCCP (100 uM). Cells were collected at the appropriate time point, washed once in PBS, trypsinized and spun. For the JC-1 staining cells were resuspended in 1 mL DMEM+10% FBS, and stained with 2 μM JC-1 dye (Molecular Probes) at 37° C. for 20 minutes in the dark. Cells were washed once and resuspended in 500 μL staining buffer (PBS+3% FBS). For Annexin V staining, cells were washed in 1× Annexin V buffer and treated as described by the Annexin V staining protocol (BD Pharmingen, San Diego, Calif.). Briefly, cells were resuspended in Annexin V buffer to a concentration of one million per mL, 100,000 cells were then stained with 5 μL of phycoerythrin (PE)-conjugated Annexin V antibody (BD Pharmingen) and 5 μL of 7-aminoactinomycin D (7AAD) and then incubated at room temperature for 15 minutes. 400 μL of Annexin V buffer was then added to each sample with gentle mixing. Stained cells were analyzed using a FACScan flow cytometer (Becton Dickinson, San Jose, Calif.). Flow cytometry data was analyzed using FlowJo 8.6 software (Tree Star Inc., Ashland, Oreg.).

Histology and Immunohistochemistry.

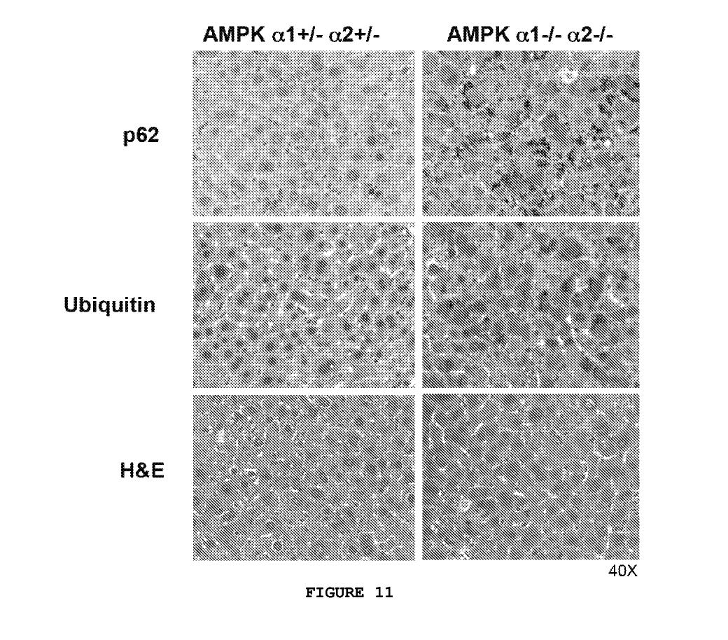

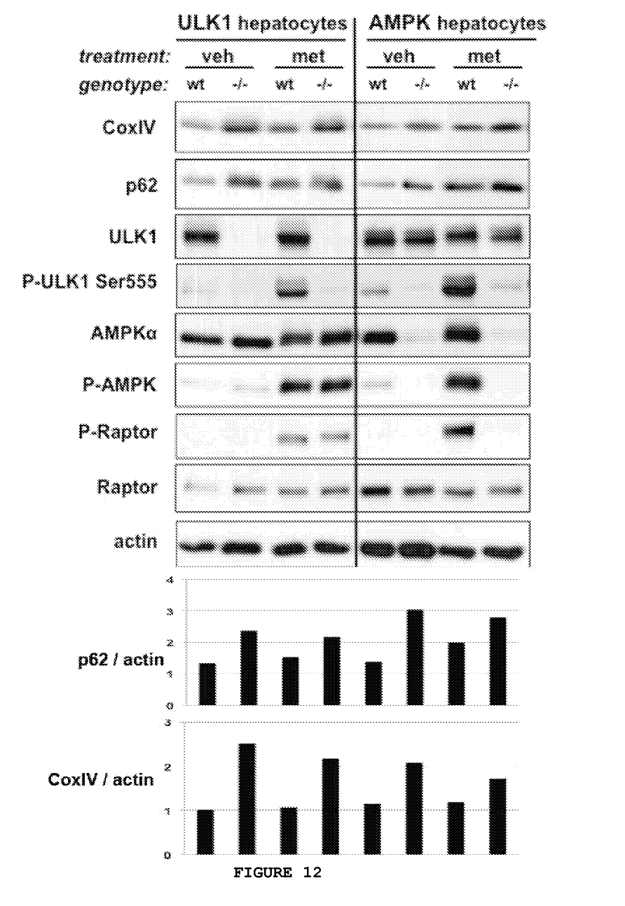

Mouse tissues were fixed in 10% formalin overnight and embedded in paraffin. For immunohistochemistry, slides were deparaffinized in xylene and ethanol and rehydrated in water. Heat mediated antigen retrieval using sodium citrate pH 6.0 buffer and slides were quenched in hydrogen peroxide (3%) to block endogenous peroxidase activity and washed in TBST buffer. Slides were blocked in 5% normal serum for 1 hr at room temperature and incubated with primary antibody diluted in blocking buffer, washed and a secondary biotinylated goat-anti mouse IgG antibody was applied. The avidin-biotin peroxidase complex method (Vector, Burlingame, Calif.) was used and staining was visualized using the DAB chromophore (Vector ABC; DAB). Slides were counterstained with hematoxylin and mounted with Fluoromount (SoutherBiotech, Birmingham, Ala.). The anti-ubiquitin (P4D1) (Cell Signaling Technology, Beverly, Mass. 1:750) and anti-p62 (Progen, Heidelberg Germany 1:200) antibodies were diluted according to manufacturer's suggestions. Images of p62 and ubiquitin shown in FIG. 11 representative of 2 independent experiments with 5 mice of each genotype analyzed.

Analysis of autophagic events in C. elegans.

The level of autophagy in various mutant strains was assessed using a GFP::LGG-1/LC3 translational reporter, which was originally constructed and later integrated (DA2123 (adIs2122[lgg1p::GFP:LGG1+ro16]). New strains used in this study were MAH14 (daf2(e1370) III; adIs2122[lgg1p::GFP:LGG1+ro16]), MAH28 (aak2(ok524) X; adIs2122[lgg1p::GFP::LGG1+ro16] and AGD383 (uthIs202[aak2 cp::AAK2 (aa1321)::TOMATO+ro16]). AGD383 crossed to DA2123 to obtain F1 heterozygous animals used for scoring. AAK-2 gain of function expression construct consisted of the 3 KB putative promoter region 5′ to the aak2c (T01C8.1c) start site driving cDNA sequence corresponding to AAK-2 aa 1-321. Expression construct backbone was based upon pPD95.77 from the Fire lab C. elegans vector kit with tdTOMATO in place of GFP. Transgenic strains were generated via microinjection of 150 ug/ul DNA into the gonad of adult hermaphrodites using standard techniques with pRF4 rol6(sul006) a transformation marker. Integrated transgenic lines were generated using gamma irradiation and outcrossed to wild type (N2) animals four times. GFP-positive foci/puncta were counted. In brief, GFP-positive foci were counted (using 1000-fold magnification on a Zeiss Axioplan II microscope) in the hypodermal seam cells of L3 transgenic animals, which were staged by gonad morphology and germline developmental phenotype. Between 3-10 seam cells were examined in each of 8-30 animals from at least two independent trials and averaged. Data analysis was done using unpaired, two-tailed t-test. When performing RNAi experiments to count GFP positive foci, young adults were fed the RNAi bacteria, and the L3 progeny of their progeny (“F2 generation”) were examined. When scoring heterozygote animals, AGD383 was raised on RNAi before being crossed, and L3 heterozygotes were subsequently analyzed. In all cases, animals were raised at 20° C.

Statistical Analysis.

Comparisons were made using the unpaired Student's t-test. SEM+/− is represented as error bars. Statistical significance as indicated.

A two-part screen was used to identify substrates of AMPK that mediate its effects on cell growth and metabolism. First, an optimal AMPK substrate motif was used to search eukaryotic databases for proteins containing conserved candidate target sites. Many in vivo substrates of AMPK not only conform to this motif, but also bind to the phospho-binding protein 14-3-3 inducibly upon phosphorylation by AMPK. Therefore proteins were screened that bound to recombinant 14-3-3 in wild-type but not AMPK-deficient cells, and only under conditions of energy stress when AMPK would be active. One protein was identified that contained multiple conserved candidate AMPK phosphorylation sites and associated with 14-3-3 in an AMPK-dependent manner was the mammalian Atg1 homolog ULK1 (FIG. 1A,B).

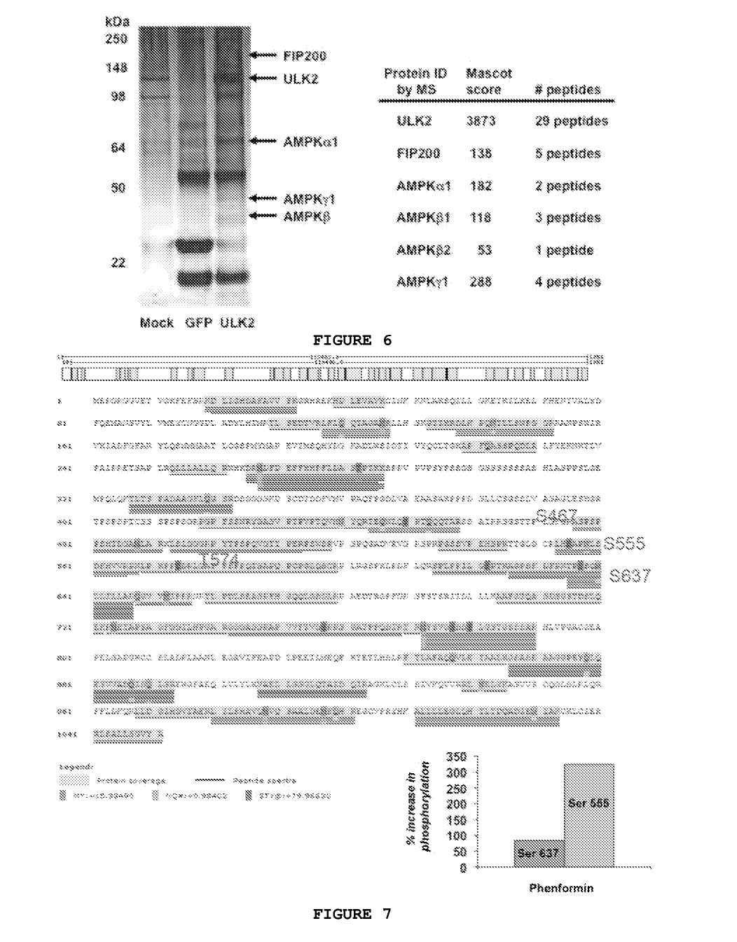

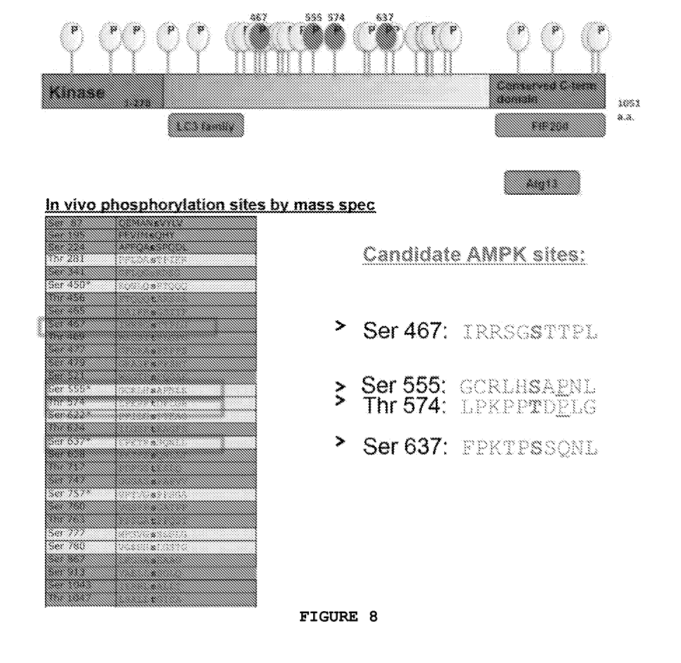

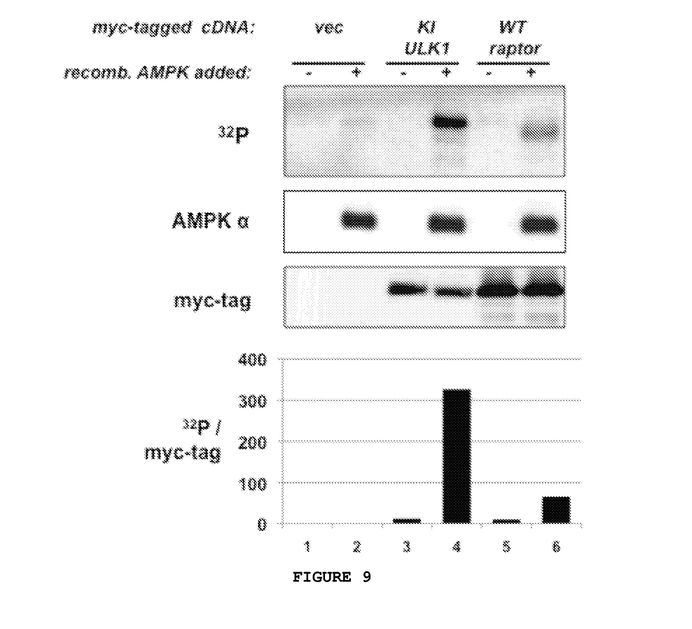

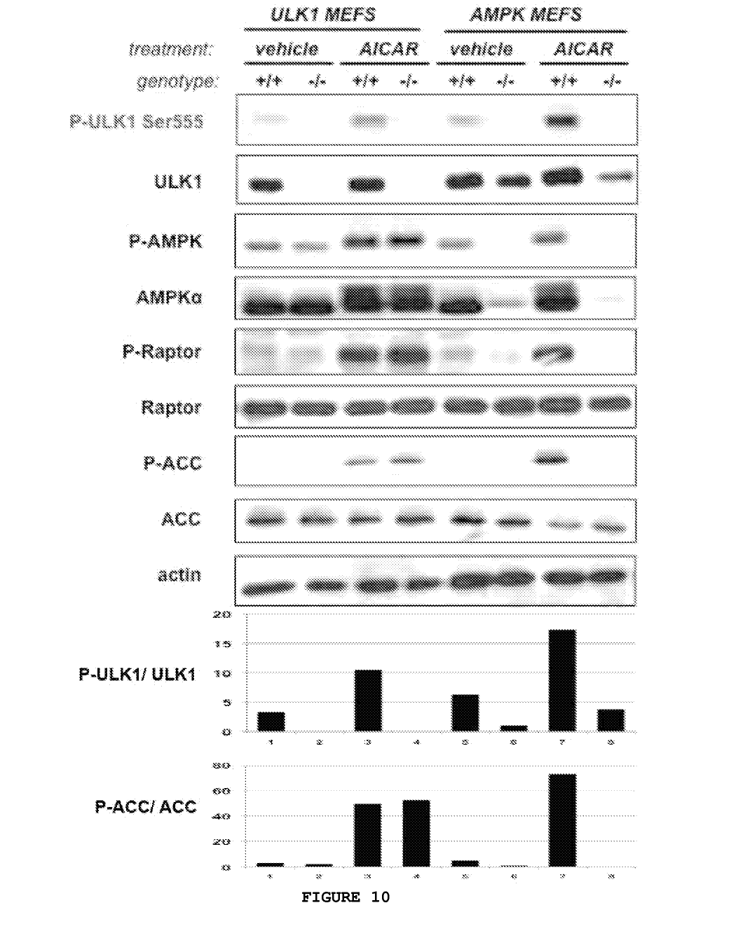

ULK1 contains four sites (Ser467, Ser555, Thr574, Ser637) matching the optimal AMPK substrate motif, all of which are conserved in higher eukaryotes. Two of the sites are conserved back to C. elegans (Ser555 and Ser574) and in the mammalian family member ULK2, though not the more distant family members ULK3 and ULK4, which unlike ULK1 and ULK2, are not thought to function in autophagy. Indeed, endogenous AMPK subunits co-immunoprecipitated with ULK1 and ULK2 but not ULK3 (FIG. 5) and AMPK subunits were found in unbiased identifications of proteins co-immunoprecipitating with overexpressed ULK2 (FIG. 6), consistent with recent proteomic analyses. To examine ULK1 in vivo phosphorylation sites, tandem mass spectrometry was used on epitope-tagged ULK1 isolated from cells treated with or without the mitochondrial complex I inhibitor phenformin. Peptides spanning three of the four candidate AMPK sites were detected in ULK1 (Ser555, Thr574, Ser637), and all three were phosphorylated only after phenformin treatment (FIG. 7, 8). To examine whether ULK1 could serve as a direct substrate for AMPK in vitro, a kinase-inactive allele (K46I) was created, to remove its autophosphorylation. AMPK phosphorylated ULK1 to a greater extent than an established substrate, Raptor (FIG. 1C, 9), which may reflect the presence of at least four potential AMPK sites in ULK1, as compared to Raptor, which has two reported AMPK sites.

Phospho-specific antibodies were generated against Ser467 and Ser555 of ULK1. Phosphorylation of both sites was induced by phenformin treatment or expression of ULK1 with a constitutively active AMPKα1 allele in the absence of energy stress (FIG. 1D). Purified AMPK also induced phosphorylation at these sites in an in vitro kinase assay, consistent with their direct phosphorylation (FIG. 1E). Using AMPK- and ULK1-deficient primary mouse embryonic fibroblasts (MEFs) or matched control wild-type MEFs, phosphorylation of endogenous ULK1 was observed on Ser555 in an AMPK-dependent manner after treatment of cells with the AMP-mimetic AICAR (FIG. 1F). Notably, the phosphorylation of ULK1 in these cells paralleled that of two bona-fide AMPK substrates, ACC and Raptor (FIG. 1F, 10).

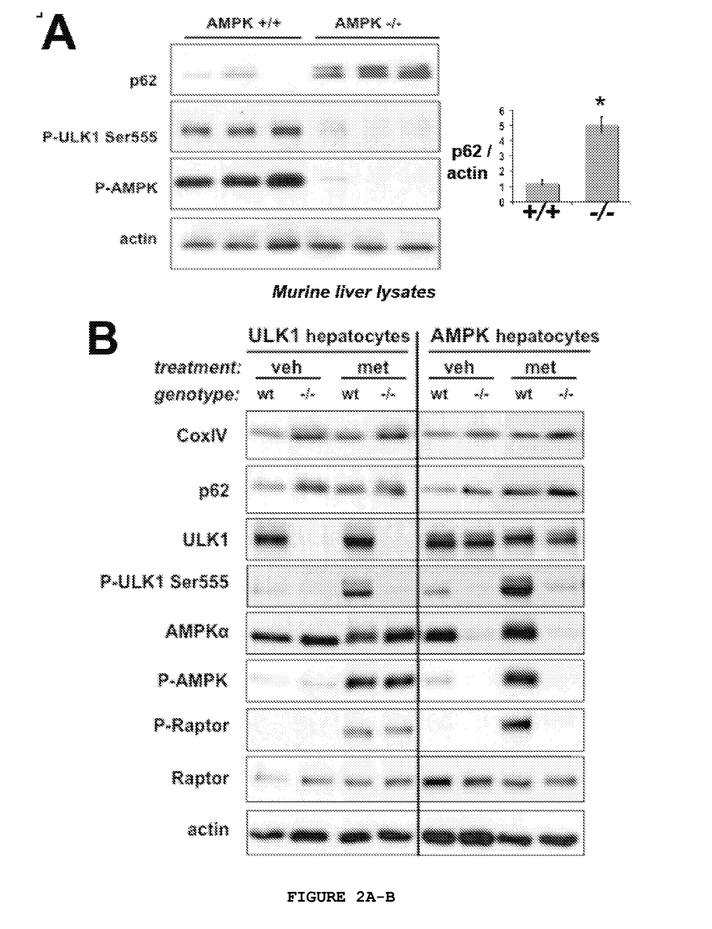

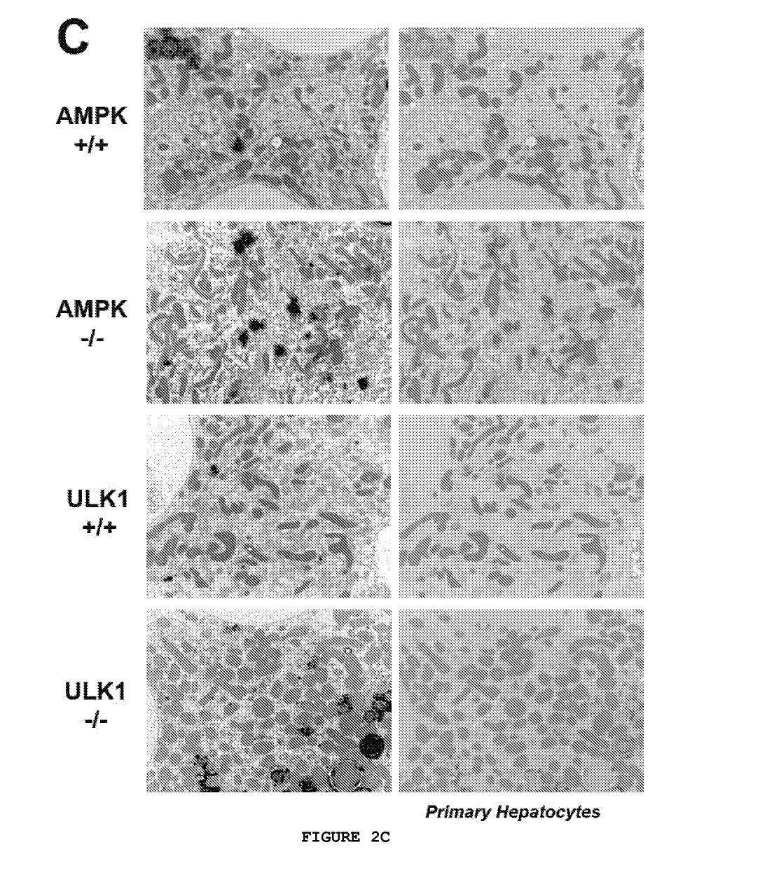

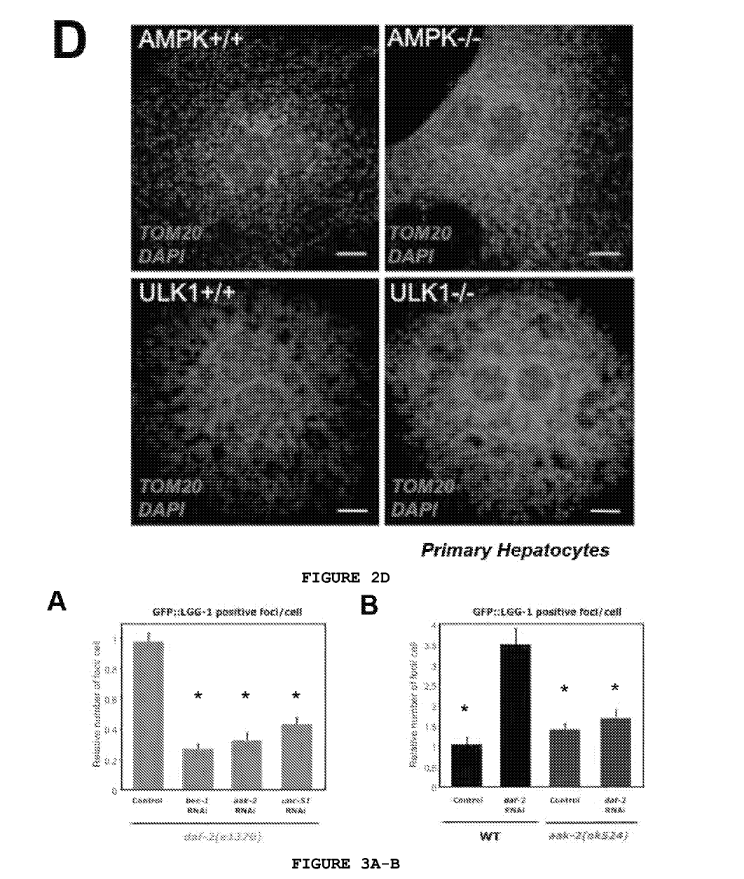

The phenotypic consequences of AMPK- or ULK1-deficiency on markers of autophagy were examined in murine liver and primary hepatocytes. Immunoblot and immunohistochemical analysis of AMPK-deficient livers showed accumulation of the p62 protein (FIG. 2A, 11), whose selective degradation by autophagy has established it as a widely used marker of this process. p62 contains a UBA ubiquitin binding domain which mediates binding to ubiquitinated cargo targeted for autophagy mediated degradation. Consistent with this function, p62 aggregates colocalized with ubiquitin aggregates in AMPK-deficient livers (FIG. 11). Notably, p62 is recruited to mitochondria targeted for mitophagy, and is involved in mitochondrial aggregation and clearance. ULK1-deficient mice exhibit accumulation of defective mitochondria in mature red blood cells, which are normally devoid of mitochondria.

Given the aberrant accumulation of p62 in the absence of AMPK in mouse liver and the fact that rodent hepatocytes undergo significant mitophagy upon culturing, it was then examined whether AMPK- or ULK1-deficiency in primary hepatocytes might exhibit mitochondrial defects. Protein levels of p62 and the mitochondrial marker protein CoxIV were similarly elevated in lysates from AMPK- or ULK1-deficient hepatocytes cells but not wild-type controls (FIG. 2B, 12). Increased phosphorylation of endogenous ULK1 Ser555 was observed in wild-type but not AMPK-deficient hepatocytes after AMPK activation by metformin treatment (FIG. 2B).

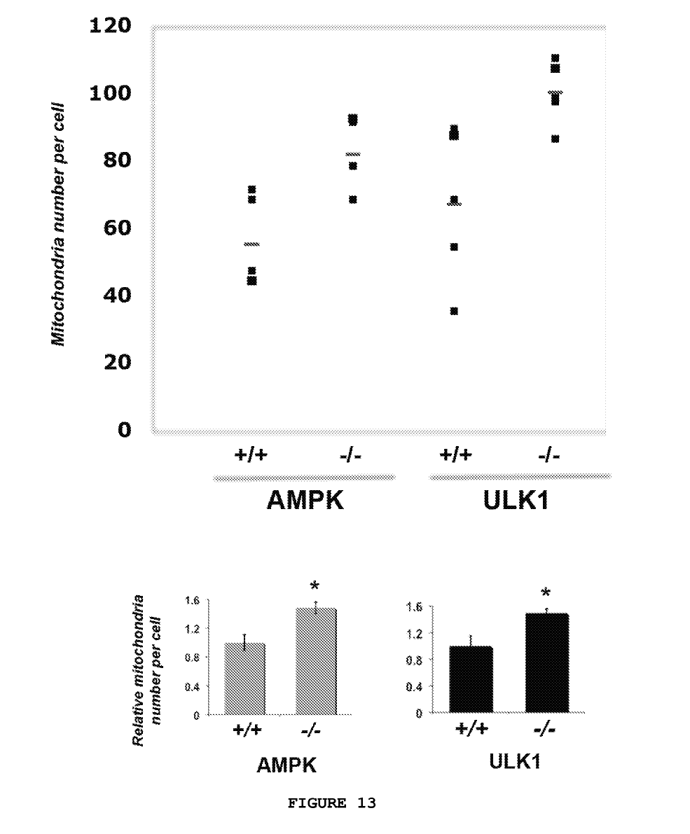

Further analysis of the ULK1 and AMPK hepatocytes using transmitting electron microscopy (TEM) revealed elevated levels of abnormal mitochondria, which was analyzed quantitatively using morphometric software (FIG. 2C, right panels). Similar to findings in other autophagy mutant hepatocytes, the number of mitochondria per cell was significantly increased in AMPK- and ULK1-deficient hepatocytes compared to wild-type controls (FIG. 13), also seen by immunocytochemical staining for the mitochondrial membrane protein TOM20 (FIG. 2D).

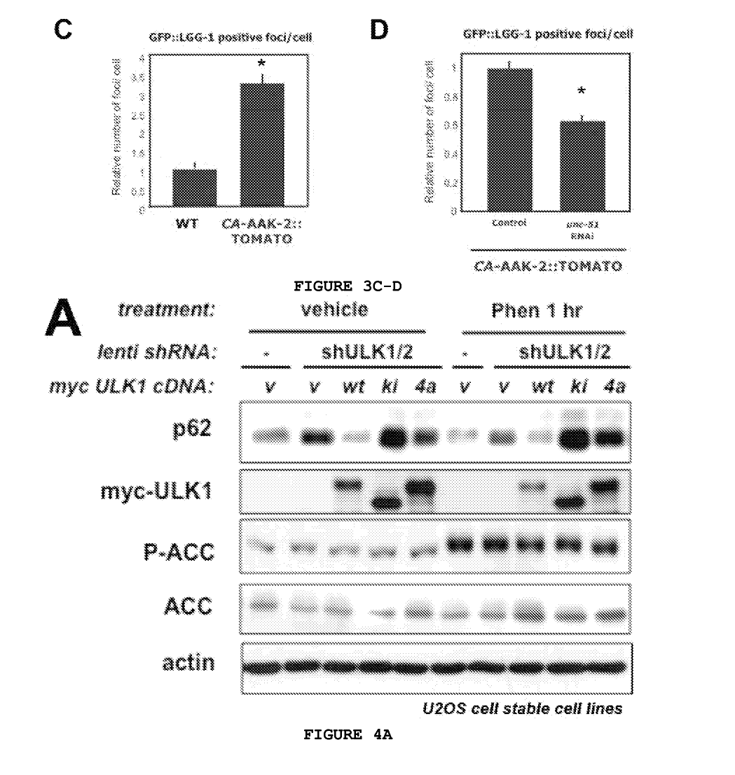

Given the conservation of AMPK sites in ULK1, a determination whether these two proteins play conserved roles in autophagy in the nematode C. elegans was then made. In a reporter assay based on the C. elegans LC3 homolog LGG-1, loss of insulin signaling was observed through genetic mutation (daf-2 (e1370)) or RNAi against the insulin receptor daf-2, resulted in increased numbers of GFP::LGG-1 positive foci in hypodermal seam cells, indicative of increased autophagy and consistent with the established role for insulin signaling in the suppression of autophagy in C. elegans. daf-2 mutant worms treated with RNAi to aak-2 or unc-51, the AMPK and ULK1 orthologs, respectively, resulted in a decrease in abundance of LGG-1 containing puncta (FIG. 3A). daf-2 RNAi failed to increase the number of LGG-1 positive foci in AMPK-deficient worms (FIG. 3B). These data indicate that both AMPK and ULK1 have critical roles in autophagy induced by reduced insulin signaling in C. elegans.

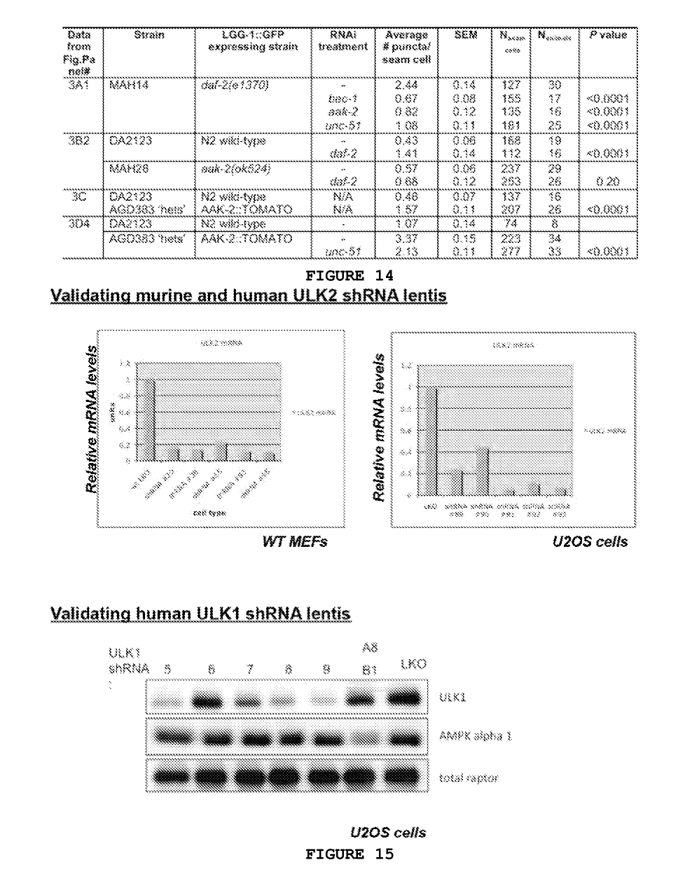

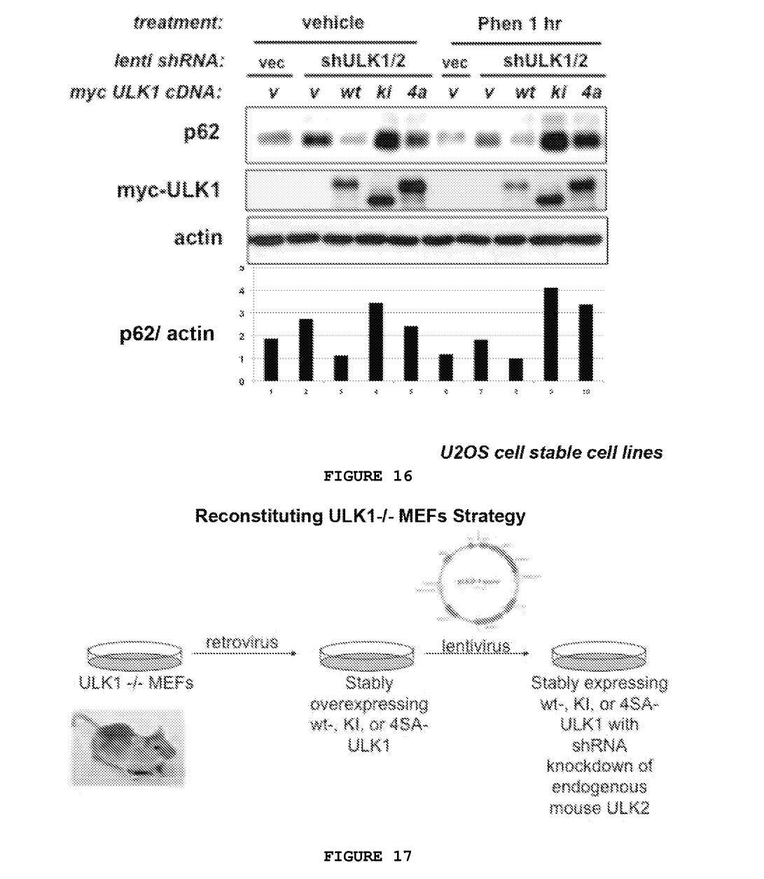

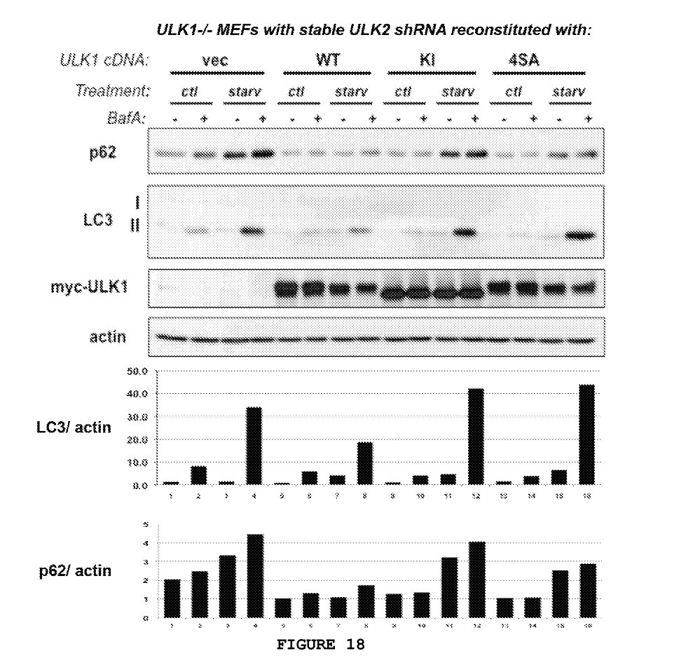

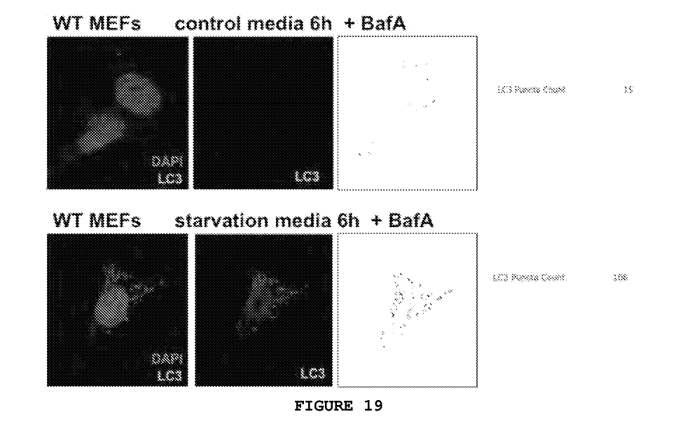

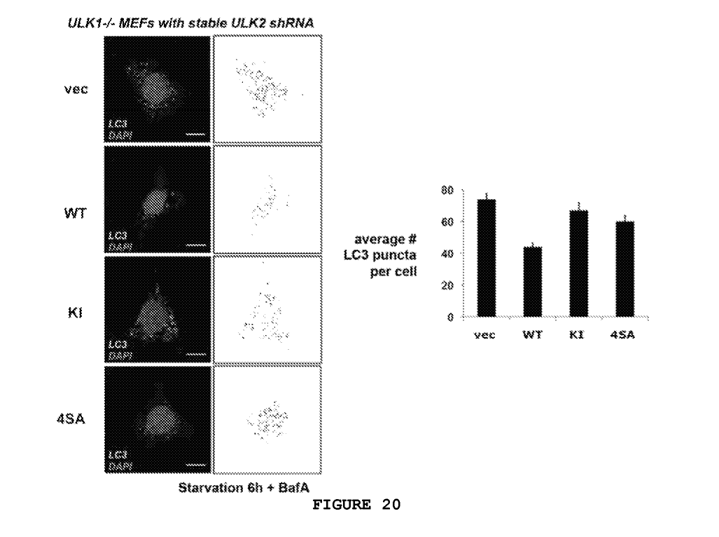

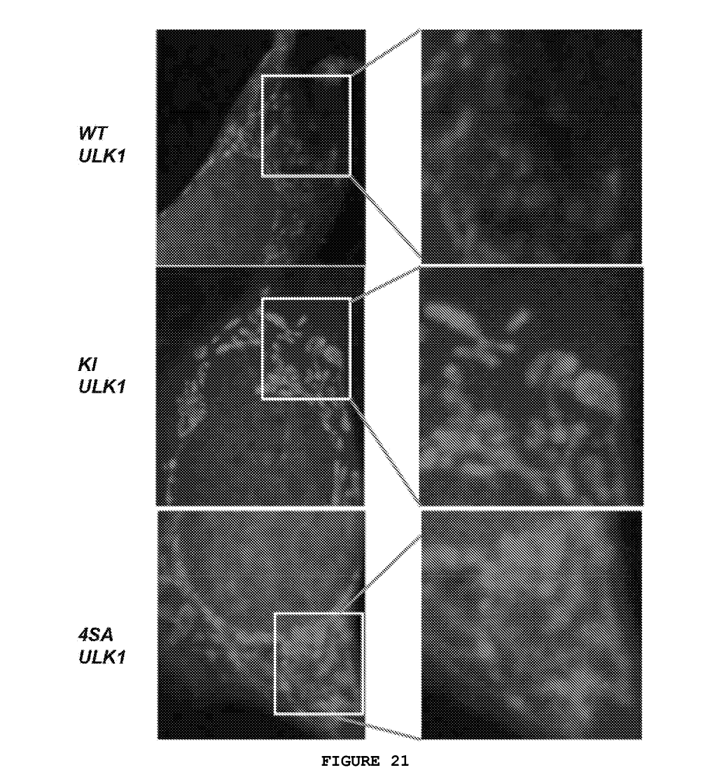

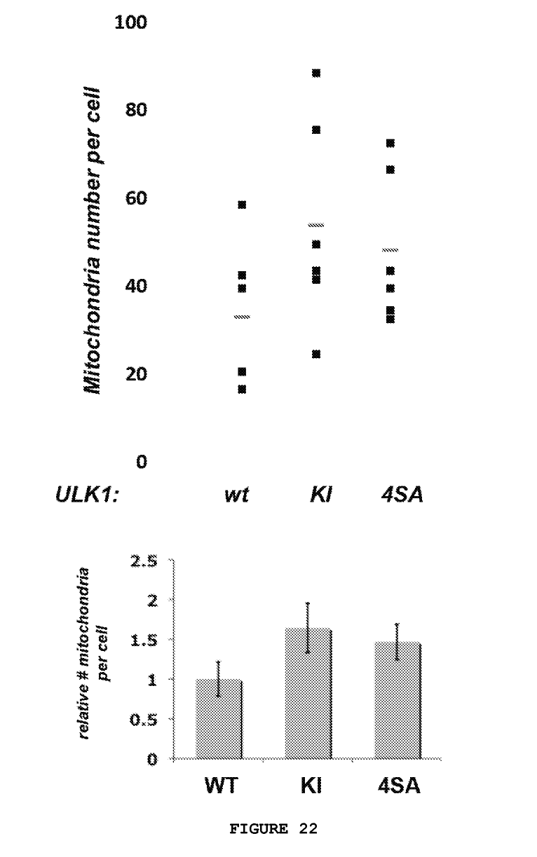

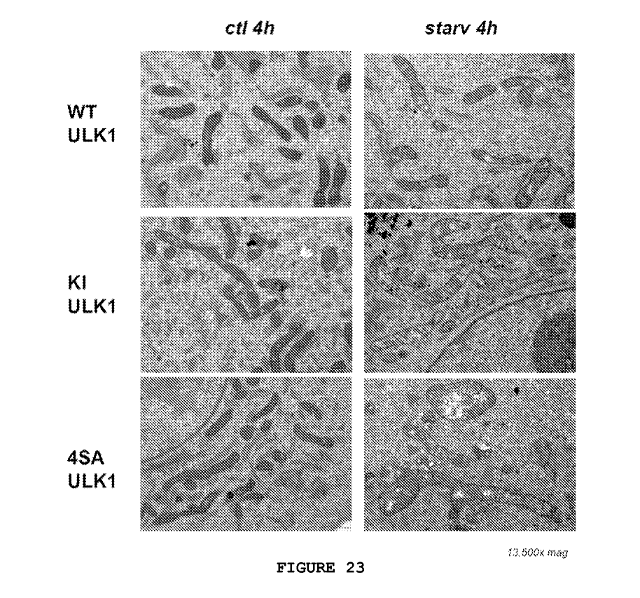

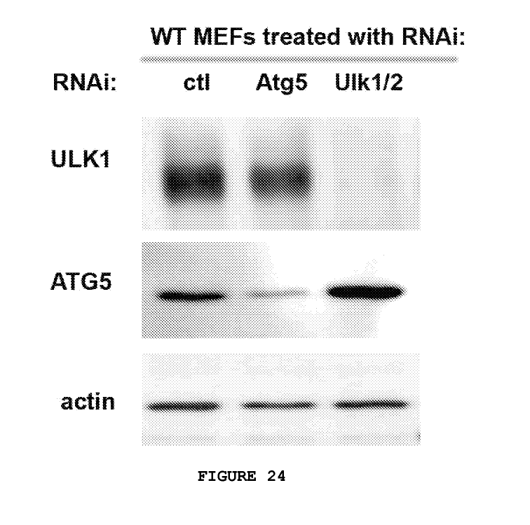

Transgenic worms expressing constitutively active AMPK exhibited a ˜3-fold increase in the number of LGG-1 positive foci in seam cells compared to the number of foci in controls (FIG. 3C). The number of LGG-1-positive foci was significantly reduced when these animals were fed unc-51 RNAi (FIG. 3D) [all raw data in FIG. 14]. These observations indicate that AMPK activation is sufficient to induce autophagy in worms, and ULK1 is required for this induction. To test whether AMPK phosphorylation of ULK1 is required for ULK1 function, wild-type (WT), catalytically inactive (KI), or the AMPK nonphosphorylatable (4SA) ULK1 cDNA was stably introduced into human osteosarcoma U2OS cells in which endogenous ULK1 and ULK2 was subsequently reduced with lentiviral hairpin shRNAs against each (FIG. 15). U2OS cells stably expressing ULK1 and ULK2 shRNA exhibited increased amounts of p62 indicative of defective autophagy compared to that of parental U2OS cells infected with an empty lentiviral vector (FIG. 4A, compare lane 1 and 2). Stable retroviral reconstitution of a myc-tagged WT ULK1 cDNA, but not the 4SA or KI mutant, restored p62 degradation (FIG. 4A, lanes 3-5; FIG. 16). Furthermore, reconstituted ULK1−/− MEFs that were also knocked down for endogenous ULK2 (FIG. 17) with WT, KI, or 4SA ULK1 cDNAs and were examined and the extent of autophagy following placement of these cell lines into starvation media. MEFs deficient for ULK1 and ULK2 contained elevated levels of p62 upon starvation. Cells reconstituted with WT ULK1 had reduced p62 levels, unlike the KI or 4SA-expressing cells which behaved like the ULK-deficient state (FIG. 4B, 18). To test whether the 4SA mutant exhibited effects on mitochondrial homeostasis, TEM and mitochondrial-selective dyes were used on the WT, KI, and 4SA ULK1 stably reconstituted ULK-deficient MEFs. TEM and Mitotracker Red staining revealed that the KI- and 4SA-ULK1 expressing cells had altered mitochondrial homeostasis compared WT ULK1 cells, denoted by increases in the overall number and aberrant morphology of mitochondria (FIG. 4C, 22, 23). The altered cristae and aberrant morphology of the mitochondria in the KI- and 4SA-ULK1 reconstituted cells was enhanced upon starvation (FIG. 23). To test whether these mitochondria were functionally impaired, the mitochondrial membrane potential was analyzed with the activity dependent JC-1 dye, which revealed defects in KI- and 4SA-reconstituted MEFs (FIG. 4D).

A hallmark of cells defective for autophagy is a predisposition to undergo apoptosis after stress stimuli that normally would activate autophagy to promote cell survival. It was then examined how ULK1/2 deficiency would compare to loss of central downstream autophagic regulator such as Atg5 in terms of requirement for cell survival following starvation. Wild-type MEFs were treated with control, Atg5, or combined ULK1 and ULK2 siRNA and analyzed for effects on cell viability after being placed into starvation conditions. Simultaneous depletion of ULK1 and ULK2 mirrored the magnitude and kinetics of cell death observed with Atg5 loss upon starvation (FIG. 4E, 24). It was then investigated whether mutation of the AMPK sites in ULK1 might also mimic ULK1/2 loss of function in this cell survival assay. ULK-deficient MEFs reconstituted with WT, but not KI or 4SA ULK1, restored cell survival after starvation (FIG. 4F). ULK1-deficient cells expressing the KI or 4SA mutant ULK1 showed rates of cell death like WT MEFs treated with Ulk1 and Ulk2 siRNA. Thus, loss of the AMPK sites in ULK1 mimics complete loss of ULK1 and ULK2 in control of cell survival after nutrient deprivation.

These findings reveal a direct connection between energy sensing and core conserved autophagy proteins. In mammals, phosphorylation of ULK1 by AMPK is required for ULK1 function in the response to nutrient deprivation. As AMPK suppresses mTOR activity and mTOR inhibits ULK1, AMPK controls ULK1 via a two-pronged mechanism, ensuring activation only under the appropriate cellular conditions (FIG. 4G, 25). There are a number of physiological and pathological contexts where this pathway is likely to play a critical role.

Beyond the conserved nature of these signaling events and the role of some autophagy genes as tumor suppressors, AMPK is defective in a variety of human cancers bearing inactivating mutations in its upstream kinase LKB1. Thus ULK1 may have a central role in the beneficial effects of the LKB1/AMPK pathway on tumor suppression or in treatment of metabolic disease, as observed here with metformin stimulation of ULK1 phosphorylation in liver and the profound defect in autophagy in AMPK-deficient livers. ULK1-dependent effects on mitochondrial homeostasis and cell survival may represent additional beneficial effects of metformin and other AMPK activators in overall organismal health and lifespan.

1. An isolated polypeptide comprising a fragment of SEQ ID NO:2 containing a phosphorylatable domain containing the sequence of SEQ ID NO:7, 8, 9 or 10, wherein the polypeptide lacks ULK1 activity.

2. An isolated polypeptide of claim 1 consisting of about 10-100 amino acids and having a phosphorylatable domain containing the sequence of SEQ ID NO:3, 4, 5, or 6.

3. The isolated polypeptide of claim 2, wherein the peptide is about 10-50 amino acids in length.

4. The isolated polypeptide of claim 2, wherein the peptide is about 10-30 amino acids in length.

5. The isolated polypeptide of claim 2, wherein the peptide is about 10-20 amino acids in length.

6. The isolated polypeptide of claim 2, wherein the peptide is about 10 amino acids in length.

7. The isolated polypeptide of any one of claims 1-6 containing at least one unnatural amino acid or D-amino acid.

8. A fusion polypeptide comprising a first domain comprising a polypeptide of any one of claims claim 1-6 and a second domain comprising a domain of interest.

9. The fusion polypeptide of claim 8, wherein the second domain is a protein transduction domain.

10. The fusion polypeptide of claim 8, wherein the second domain is a receptor ligand domain.

11. A method of screening for an agent that modulates autophagy or energy metabolism comprising contacting a polypeptide of any of claims 1-6 with an agent in the presence of an AMPK polypeptide and determining whether the peptide is phosphorylated or dephosphorylated, wherein a change in phosphorylation compared to the polypeptide in the presence of AMPK but in the absence of the agent is indicative of an agent that modulates autophagy.

12. The method of claim 10, wherein the agent is selected from the group consisting of a peptide, peptidomimetic, polypeptide, antibody, antibody fragment or small molecule.

13. An isolated phosphorylation site-specific antibody that specifically binds to a human ULK1 polypeptide antigenic domain at a site containing a peptide of claim 6.

14. An isolated phosphorylation site-specific antibody of claim 13 that specifically binds a human ULK1 polypeptide at a sequence selected from the group consisting of SEQ ID NO:3, 4, 5, or 6, wherein said antibody binds said polypeptide when phosphorylated at the serine of SEQ ID NO:3, 5, or 6 or the threonine of SEQ ID NO:5.

15. The isolated phosphorylation site specific antibody of claim 14, wherein the sequence is SEQ ID NO:3 and the phosphorylated residue is the serine.

16. An isolated phosphorylation site-specific antibody of claim 13 that specifically binds a human ULK1 polypeptide at a sequence selected from the group consisting of SEQ ID NO:3, 4, 5, or 6, wherein said antibody does binds said polypeptide when not phosphorylated at the serine of SEQ ID NO:3, 4, or 6 or the threonine of SEQ ID NO:5.

16. A method of detecting a cancer associated with aberrant autophagy comprising contacting a cancer tissue sample with an antibody of claim 13 and determining whether the antibody binds to a ULK polypeptide in the sample, wherein binding of a non-phosphorylated ULK1 is indicative of a cancer associated with aberrant autophagy.

17. A method of increasing a cellular response to a cancer therapy comprising inhibiting phosphorylation or activity of ULK1 in a cell currently undergoing the cancer therapy.

18. The method of claim 17, wherein said inhibiting comprises contacting the cell with an agent capable of inhibiting phosphorylation.

19. The method of claim 17, wherein said inhibiting comprises inhibiting phosphorylation at a site containing a sequence selected from the group consisting of SEQ ID NO: 3, 4, 5, or 6.

20. The method of claim 17, wherein the method comprises contacting a cancer cell with a polypeptide of any one of claim 1-6.

21. The method of claim 17, wherein the cancer therapy comprises endocrine therapy, chemotherapy or radiation therapy.

22. The method of claim 21 wherein the cancer therapy comprises administration of tamoxifen or a related taxane.

23. A method of treating, inhibiting or preventing a cancer, type II diabetes, inflammatory disease or mental disease or disorder associated with protein misfolding and aggregation comprising contacting a cell with an agent that promotes phosphorylation of ULK1 at a site containing a sequence selected from the group consisting of 3, 4, 5, or 6.

24. The method of claim 17 or 23, wherein the cancer is breast cancer, liver cancer, ovarian cancer, gastric cancer, bladder cancer, colon cancer, prostate cancer, lung cancer, nasopharyngeal carcinoma, cervical carcinoma, skin cancer, brain cancer, neuroblastoma, glioma, a solid tumour, a hematologic malignancy, leukemia, lymphoma, or head and neck cancer.

25. The method of claim 23, wherein the mental disease or disorder associated with protein misfolding or aggregation is selected from the group consisting of Alzheimer disease, Parkinson disease, tauopathies, and polyQ3 expansion diseases.

26. A pharmaceutical composition comprising a modulator of phosphorylation of ULK 1 at a site containing the sequence of SEQ ID NO:3, 4, 5, or 6.


SEQUENCE LISTING (12 sequences)

SEQ ID NO: 1 - DNA, 3156 bp, Mus musculus
  CDS (1)..(3156)
atggagccgggccgcggcggcgtcgagaccgtgggcaagttcgagttctctcgcaaggacctgattggacacggcgccttcgcggtggtcttcaagggtcgacaccgcgagaagcacgacctggaggtggccgtcaaatgcattaacaagaagaaccttgccaagtcccaaacactgctgggaaaggaaatcaaaatcctgaaggaactaaagcacgaaaacatcgtggcgctgtatgacttccaggaaatggctaattctgtctacctggtcatggagtattgtaatggtggagacctggctgactacctgcacactatgcgcacactgagtgaagacactgtcaggcttttcctacagcagatcgctggcgccatgcggctgctgcacagcaagggcatcatccaccgggacctgaagccccaaaacatcctgctgtccaaccctgggggccgccgggccaaccccagcaacatccgagtcaagattgctgactttggattcgctcggtacctccagagcaacatgatggcggccacactctgtggttctcctatgtacatggctcctgaggtcattatgtcccagcactacgatggaaaggctgacctgtggagcattggcaccattgtctaccagtgtctgacagggaaggccccttttcaggccagcagccctcaggatttgcgcctgttttatgagaagaacaagacactagttcctgccatcccccgggagacatcagctcccctgcggcagctgctcctggctctgttgcagcggaaccacaaggaccgcatggactttgatgaatttttccaccaccctttcttggatgccagcacccccatcaagaaatccccacctgtgcctgtgccctcatatccaagctcagggtctggcagcagctccagcagcagctctgcctcccacctggcctctccaccgtccctgggggagatgccacagctacagaagacccttacctccccagccgatgctgctggctttcttcagggctcccgggactctggtggcagcagcaaagactcctgtgacacagatgactttgtcatggtcccagcccagtttccaggtgatctagttgctgaggcagccagtgccaagcccccacctgatagcctgctgtgtagtgggagctcattggtggcctctgctggcctagagagccacggccgtaccccctctccctctccgacctgcagcagctctcccagcccctctggccggcctggccccttctccagcaacaggtacggtgcctcggtccccattcctgtccccactcaggtgcacaattaccagcgcatcgagcaaaacctgcaatcgcccactcaacagcagacagccaggtcctctgccatccgaaggtcagggagcaccagccccctgggctttggccgggccagcccatcacccccctcccacaccgatggagccatgctggccaggaagctgtcacttggaggtggccgtccctacacaccttctccccaagtgggaaccatcccagagcgacccagctggagcagagtgccctccccacaaggagctgatgtgcgggttggcaggtcaccacgacccggttcctctgtgcctgagcactctccaagaaccactgggctgggctgccgcctgcacagtgcccctaacctgtccgacttccatgttgtgcgtcccaagctgcctaagcccccaacagacccactgggagccacctttagcccaccccagaccagcgcaccccagccatgcccagggctacagtcttgccggccactgcgtggctcacctaagctgcctgacttcctacagcggagtcccctaccccccatcctaggctctcctaccaaggccgggccctcctttgacttccccaaaacccccagctctcagaatttgctgaccctgttggctaggcagggggtagtaatgacaccacctcggaaccgtacactgcctgacctctccgaggccagtcctttccatggccagcagctgggctctggccttcggcccgctgaagacacccggggtccctttggacggtccttcagcaccagccgcattacggacctgctgcttaaggctgcatttgggactcaggcctctgactcaggcagcacagacagcctacaggagaaacctatggagattgctccctctgctggctttggagggactctgcatccaggagctcgtggtggaggggccagcagcccagcacctgtggtatttactgtaggctccccacccagtggtgccaccccaccccagagtacccgtaccagaatgttctcagtgggctcttccagctccctgggctctactggctcctcctctgcccgccacttagtgcctggggcctgtggagaggccccggagctttctgccccaggccactgctgtagccttgctgacccccttgctgccaacttggagggggctgtgaccttcgaggctcctgacctcccagaggagaccctcatggagcaagagcacacggaaaccctacacagtctgcgcttcacactagcgtttgcacagcaagttctggagattgcagccctgaagggaagtgccagtgaggccgccggtggccctgagtaccagctccaggaaagtgtggtggctgaccagatcagtcagttgagccgagagtggggctttgcagagcaactggttctgtacttgaaggtggctgagctgctgtcctcaggcctacagactgccattgaccagattcgagctggcaaactctgcctttcatctactgtgaagcaggtggtacgcagactaaatgagctgtacaaggccagcgtggtatcctgccagggcctcagcttgcgacttcagcgcttctttctggacaaacaacggctgctggacgggatccatggtgtcactgcagagcggctcatcctcagccatgctgtgcaaatggtacaatcagctgcccttgatgagatgttccagcaccgagagggctgtgtaccgagatatcacaaagccctgctattgctggaggggttgcagcacactctcacggaccaggcagacattgagaacattgccaaatgcaagctgtgcattgagaggagactctcggccctgctgagtggtgtctatgcctga

SEQ ID NO: 2 - protein, 1051 aa, Mus musculus
MEPGRGGVETVGKFEFSRKDLIGHGAFAVVFKGRHREKHDLEVAVKCINKKNLAKSQTLLGKEIKILKELKHENIVALYDFQEMANSVYLVMEYCNGGDLADYLHTMRTLSEDTVRLFLQQIAGAMRLLHSKGIIHRDLKPQNILLSNPGGRRANPSNIRVKIADFGFARYLQSNMMAATLCGSPMYMAPEVIMSQHYDGKADLWSIGTIVYQCLTGKAPFQASSPQDLRLFYEKNKTLVPAIPRETSAPLRQLLLALLQRNHKDRMDFDEFFHHPFLDASTPIKKSPPVPVPSYPSSGSGSSSSSSSASHLASPPSLGEMPQLQKTLTSPADAAGFLQGSRDSGGSSKDSCDTDDFVMVPAQFPGDLVAEAASAKPPPDSLLCSGSSLVASAGLESHGRTPSPSPTCSSSPSPSGRPGPFSSNRYGASVPIPVPTQVHNYQRIEQNLQSPTQQQTARSSAIRRSGSTSPLGFGRASPSPPSHTDGAMLARKLSLGGGRPYTPSPQVGTIPERPSWSRVPSPQGADVRVGRSPRPGSSVPEHSPRTTGLGCRLHSAPNLSDFHVVRPKLPKPPTDPLGATFSPPQTSAPQPCPGLQSCRPLRGSPKLPDFLQRSPLPPILGSPTKAGPSFDFPKTPSSQNLLTLLARQGVVMTPPRNRTLPDLSEASPFHGQQLGSGLRPAEDTRGPFGRSFSTSRITDLLLKAAFGTQASDSGSTDSLQEKPMEIAPSAGFGGTLHPGARGGGASSPAPVVFTVGSPPSGATPPQSTRTRMFSVGSSSSLGSTGSSSARHLVPGACGEAPELSAPGHCCSLADPLAANLEGAVTFEAPDLPEETLMEQEHTETLHSLRFTLAFAQQVLEIAALKGSASEAAGGPEYQLQESVVADQISQLSREWGFAEQLVLYLKVAELLSSGLQTAIDQIRAGKLCLSSTVKQVVRRLNELYKASVVSCQGLSLRLQRFFLDKQRLLDGIHGVTAERLILSHAVQMVQSAALDEMFQHREGCVPRYHKALLLLEGLQHTLTDQADIENIAKCKLCIERRLSALLSGVYA

SEQ ID NO: 3 - protein, 10 aa, Artificial sequence
  phosphorylation domain of ULK1
IRRSGSTTPL

SEQ ID NO: 4 - protein, 10 aa, Artificial sequence
  phosphorylation domain of ULK1
GCRLHSAPNL

SEQ ID NO: 5 - protein, 10 aa, Artificial Sequence
  phosphorylation domain of ULK1
LPKPPTDPLG

SEQ ID NO: 6 - protein, 10 aa, Artificial Sequence
  phosphorylation domain of ULK1
FPKTPSSQNL

SEQ ID NO: 7 - protein, 10 aa, Artificial sequence
  Phosphorylation consensus sequence
LRRVXSXXNL

SEQ ID NO: 8 - protein, 10 aa, Artificial sequence
  Phosphorylation consensus sequence
MKKSXSXPDV

SEQ ID NO: 9 - protein, 10 aa, Artificial Sequence
  Phosphorylation consensus sequence
IXHRXSXXEI

SEQ ID NO: 10 - protein, 10 aa, Artificial Sequence
  Phosphorylation consensus sequence
GXRLXSAPXL

SEQ ID NO: 11 - DNA, 3153 bp, Homo sapiens
  CDS (1)..(3153)
atggagcccggccgcggcggcacagagaccgtgggcaagttcgagttctcccgcaaggacctgatcggccacggcgccttcgcggtggtcttcaagggccgccaccgcgagaagcacgatttggaggtcgccgtcaagtgcattaacaagaagaacctcgccaagtctcagacgctgctggggaaggaaatcaaaatcctgaaggaactgaaacatgaaaacatcgtggccctgtacgacttccaggaaatggctaattctgtctacctggttatggagtactgcaacggtggggacctggccgactacctgcacgccatgcgcacgctgagcgaggacaccatcaggctcttcctgcagcagatcgcgggcgccatgcggcttctgcacagcaaaggcatcatccaccgcgacctgaaaccgcagaacatcctgctgtccaaccccgccggccgccgcgccaaccccaacagcatccgcgtcaagatcgctgacttcggcttcgcgcggtacctccagagcaacatgatggcggccacactctgcggctcccccatgtacatggcccccgaggtcatcatgtcccagcactacgacgggaaggcggacctgtggagcatcggcaccatcgtctaccagtgcctgacggggaaggcgcccttccaggccagcagcccccaggacctgcgcctgttctacgagaagaacaagacgttggtccccaccatcccccgggagacctcggccccgctgcggcagctgctcctggccctactgcaacgcaaccacaaggaccgcatggacttcgatgagttttttcatcaccctttcctcgatgccagcccctcggtcaggaaatccccacccgtgcctgtgccctcgtacccaagctcggggtccggcagcagctccagcagcagctccacctcccacctggcctccccgccgtccctgggcgagatgcagcagctgcagaagaccctggcctccccggctgacaccgctggcttcctgcacagctcccgggactctggtggcagcaaggactcttcctgtgacacagacgacttcgtcatggtccccgcgcagtttccaggtgacctggtggctgaggcgcccagtgccaaacccccgccagacagcctgatgtgcagtgggagctcactggtggcctctgcgggcttggagagccacggccggaccccatctccatccccaccctgcagcagctcccccagtccctcaggccgggctggcccgttctccagcagcaggtgcggcgcctctgtccccatcccagtccccacgcaggtgcagaactaccagcgcattgagcgaaacctgcagtcacccacccagttccaaacacctcggtcctctgccatccgcaggtcaggcagcaccagccccctgggctttgcaagggccagcccctcgccccctgcccacgctgagcatggaggcgtcctggccaggaagatgtctctgggtggaggccggccctacacgccatctcctcaagttggaaccatccctgagcggccaggctggagcgggacgccctccccacagggagctgagatgcggggtggcaggtcccctcgtccaggctcctctgcacccgagcactctccccgcacttccgggctgggctgccgcctgcacagcgcccccaacctgtctgacttgcacgtcgtccgccccaagctgcccaaaccccccacggaccccctgggagctgtgttcagcccaccacaggccagccctccccagccgtcccacggcctgcagtcctgccggaacctgcggggctcacccaagctgcccgacttcctgcagcgaaaccccctgccccccatcctgggctcccccaccaaggctgtgccctcctttgacttcccgaagacccccagctcccagaacctgctggccctcctagcccggcagggcgtggtgatgacgccccctcgaaaccggacgctgcccgacctctcggaggtgggacccttccatggtcagccgttgggccctggcctgcggccaggcgaggaccccaagggcccctttggccggtctttcagcaccagccgcctcactgacctgctccttaaggcggcgtttgggacacaagccccggacccgggcagcacggagagcctgcaggagaagcccatggagatcgcaccctcagctggctttggagggagcctgcacccaggagcccgtgctgggggcaccagcagcccttccccggtggtcttcaccgtgggctctcccccgagcgggagcacgcccccccagggcccccgcaccaggatgttctcagcgggccccactggctctgccagctcttctgcccgccacctggtgcctgggccctgcagcgaggccccagcccctgagctccctgctccaggacacggctgcagctttgccgaccccattgctgcgaacctggagggggctgtgaccttcgaggcccccgacctccctgaggagaccctcatggagcaagagcacacggagatcctgcgtggcctgcgcttcacgctgctgttcgtgcagcacgtcctggagatcgcagccctgaagggcagcgccagtgaggcggcggggggccctgagtaccagctgcaggagagtgtggtggccgaccagatcagcctgctgagccgagaatggggcttcgcggaacagctggtgctgtacctgaaggtggccgagctactgtcctccggcctgcaaagtgccatcgaccagatccgggccggcaagctctgcctgtcgtccactgtgaagcaggtggtgcgcaggctgaatgagctgtacaaggccagcgtggtgtcctgccagggcctgagcctgcggctgcagcgcttcttcctggacaagcagcggctcctggaccgcattcacagcatcactgccgagaggctcatcttcagccacgctgtgcagatggtgcagtcggctgccctggacgagatgttccagcaccgtgagggctgcgtcccacgctaccacaaggccctgctgctcctggaggggctgcagcacatgctctcggaccaggccgacatcgagaacgtcaccaagtgcaagctgtgcattgagcggagactctcggcgctgctgactggcatctgtgcctga

SEQ ID NO: 12 - protein, 1050 aa, Homo sapiens
MEPGRGGTETVGKFEFSRKDLIGHGAFAVVFKGRHREKHDLEVAVKCINKKNLAKSQTLLGKEIKILKELKHENIVALYDFQEMANSVYLVMEYCNGGDLADYLHAMRTLSEDTIRLFLQQIAGAMRLLHSKGIIHRDLKPQNILLSNPAGRRANPNSIRVKIADFGFARYLQSNMMAATLCGSPMYMAPEVIMSQHYDGKADLWSIGTIVYQCLTGKAPFQASSPQDLRLFYEKNKTLVPTIPRETSAPLRQLLLALLQRNHKDRMDFDEFFHHPFLDASPSVRKSPPVPVPSYPSSGSGSSSSSSSTSHLASPPSLGEMQQLQKTLASPADTAGFLHSSRDSGGSKDSSCDTDDFVMVPAQFPGDLVAEAPSAKPPPDSLMCSGSSLVASAGLESHGRTPSPSPPCSSSPSPSGRAGPFSSSRCGASVPIPVPTQVQNYQRIERNLQSPTQFQTPRSSAIRRSGSTSPLGFARASPSPPAHAEHGGVLARKMSLGGGRPYTPSPQVGTIPERPGWSGTPSPQGAEMRGGRSPRPGSSAPEHSPRTSGLGCRLHSAPNLSDLHVVRPKLPKPPTDPLGAVFSPPQASPPQPSHGLQSCRNLRGSPKLPDFLQRNPLPPILGSPTKAVPSFDFPKTPSSQNLLALLARQGVVMTPPRNRTLPDLSEVGPFHGQPLGPGLRPGEDPKGPFGRSFSTSRLTDLLLKAAFGTQAPDPGSTESLQEKPMEIAPSAGFGGSLHPGARAGGTSSPSPVVFTVGSPPSGSTPPQGPRTRMFSAGPTGSASSSARHLVPGPCSEAPAPELPAPGHGCSFADPIAANLEGAVTFEAPDLPEETLMEQEHTEILRGLRFTLLFVQHVLEIAALKGSASEAAGGPEYQLQESVVADQISLLSREWGFAEQLVLYLKVAELLSSGLQSAIDQIRAGKLCLSSTVKQVVRRLNELYKASVVSCQGLSLRLQRFFLDKQRLLDRIHSITAERLIFSHAVQMVQSAALDEMFQHREGCVPRYHKALLLLEGLQHMLSDQADIENVTKCKLCIERRLSALLTGICA